(19) United States
(12) Patent Application Publication
(10) Pub. No.: US 2024/0390515 A1
(43) Pub. Date: Nov. 28, 2024

(54) MULTIDOSE OPHTHALMIC COMPOSITIONS

(71) Applicant: Oculis Operations Sàrl, Lausanne (CH)

(72) Inventors: Thorsteinn Loftsson, Reykjavik (IS); Frédéric Pilotaz, Nice (FR)

(73) Assignee: Oculis Operations Sàrl, Lausanne (CH)

(21) Appl. No.: 18/792,377

(22) Filed: Aug. 1, 2024

Related U.S. Application Data
(63) Continuation of application No. PCT/EP2023/052472, filed on Feb. 1, 2023.

(30) Foreign Application Priority Data
Feb. 2, 2022 (EP) 22154808.4

(51) Int. Cl. A61K 47/69; A61K 9/00; A61K 9/08; A61K 47/02; A61K 47/10; A61K 47/12; A61K 47/18

(52) U.S. Cl. CPC A61K 47/6951; A61K 9/0048; A61K 9/08; A61K 47/02; A61K 47/10; A61K 47/12; A61K 47/183

(57) ABSTRACT

The present invention relates to aqueous cyclodextrin compositions containing drug/cyclodextrin complexes and sorbic acid or a pharmaceutically acceptable sorbate that exhibit a superior antimicrobial preservation effect at a low preservative concentration, and to an aqueous eye drop microsuspension containing solid drug/cyclodextrin complexes and sorbic acid or a pharmaceutically acceptable sorbate, which is suitable as a multidose formulation.

### CROSS REFERENCE TO RELATED APPLICATIONS

This application is a continuation of International Patent Application No. PCT/EP2023/052472, filed in the European Receiving Office on Feb. 1, 2023, which claims the benefit of European Patent Application No. 22154808.4, filed Feb. 2, 2022. The entirety of each of these applications is hereby incorporated by reference for all purposes.

### FIELD

The present disclosure relates to aqueous cyclodextrin compositions containing drug/cyclodextrin complexes and sorbic acid or a pharmaceutically acceptable sorbate that exhibit a superior antimicrobial preservation effect at a low preservative concentration, and to an aqueous eye drop microsuspension containing solid drug/cyclodextrin complexes and sorbic acid or a pharmaceutically acceptable sorbate, which is suitable as a multidose formulation.

### BACKGROUND

A major obstacle to the pharmaceutical application of cyclodextrins is the inactivation of antimicrobial preservatives agent through complexation by cyclodextrins. This is more specifically challenging for liquid aqueous based compositions such as those used for topical delivery to either the skin or the ocular surface. Only the non-complexed and unbound fraction of a preservative is able to interact with microorganisms. The fraction of the preservative that is complexed by cyclodextrin or solid microparticles is inactive from a microbiological standpoint.

In liquid pharmaceutical compositions the preservative concentrations should be kept relatively low and generally well below 1% (w/v). It has been shown that relatively lipophilic preservatives that have a somewhat high affinity for cyclodextrins are inactivated even at low cyclodextrin concentrations, while the activity of more hydrophilic preservatives that have less affinity for cyclodextrins is less affected (Thorsteinn Loftsson, Ólöf Stefánsdóttir, Hafrún Fridriksdóttir and Örn Gudmundsson, Interactions between preservatives and 2-hydroxypropyl-β-cyclodextrin, Drug Dev. Indust. Pharm. 18 (13), 1477-1484, 1992).

Since Cyclodextrins inactivate antimicrobial preservatives by forming preservative/cyclodextrin complexes, the preservative concentration needs to be increased in order to maintain the antimicrobial effect (Thorsteinn Loftsson, Ólöf Stefánsdóttir, Hafrún Fridriksdóttir and Örn Gudmundsson, Interactions between preservatives and 2-hydroxypropyl-β-cyclodextrin, Drug Dev. Indust. Pharm. 18 (13), 1477-1484, 1992; Koichiro Miyajima, Masatoshi Ikuto, Masayuki Nakagaki, Interaction of short-chain alkylammonium salts with cyclodextrins in aqueous-solutions, Chemical & Pharmaceutical Bulletin, 35 (1), 389-393, 1987).

The higher affinity a given antimicrobial preservative has for the cyclodextrin, the greater surplus of preservative is needed to maintain the preservative efficacy (René Holm, Niels Erik Olesen, Signe Dalgaard Alexandersen, Birgitte N. Dahlgaard, Peter Westh, Huiling Mu, Thermodynamic investigation of the interaction between cyclodextrins and preservatives-Application and verification in a mathematical model to determine the needed preservative surplus in aqueous cyclodextrin formulations, European Journal of Pharmaceutical Sciences, 87, 22-29, 2016).

However, there is a regulatory upper concentration limit for preservatives in ophthalmic drug formulations. In general, hydrophilic preservatives have lower affinity for cyclodextrins than lipophilic ones. Thus, hydrophilic preservatives are preferred in aqueous pharmaceutical formulations that contain cyclodextrins as solubilizers.

Several multi-dose cyclodextrin-containing eye drop formulations containing antimicrobial preservatives have been marketed, see Table 1.

TABLE 1
Aqueous multi-dose eye drop formulations that use cyclodextrin as solubilizer.

| Drug | Cyclodextrin | Preservative |
|---|---|---|
| Chloramphenicol (Clorocil ®, Edol, EU) | Methylated β-cyclodextrin | Benzalkonium chloride |
| Diclofenac (Voltaren ®, Novartis, EU) | 2-Hydroxypropyl-γ-cyclodextrin | Benzalkonium chloride |
| Indomethacin (Indocid ®, Chauvin, EU) | 2-Hydroxypropyl-β-cyclodextrin | Sodium mercurothiolate ^{a)} |
| Thimerosal (Vitaseptol ®, Novartis, EU | β-Cyclodextrin | Thiomersal ^{a)} |

^{a)} Preservatives containing mercury are no longer allowed in ophthalmic products.
Clorocil ® eye drops contain chloramphenicol (0.8% w/v) in a vehicle of boric acid, borax, benzalkonium chloride (0.01% w/v), dimethyl-β-cyclodextrin and sodium chloride in purified water. Chloramphenicol is a broad-spectrum antibiotic that is effective in treating ocular infections such as conjunctivitis, blepharitis etc. caused by a number of bacteria. Thus, the preservative used in Clorocil ® consist of the active ingredient itself and benzalkonium chloride.
Voltaren ® eye drops contain diclofenac sodium (0.1% w/v) in a vehicle of benzalkonium chloride (0.005% w/v), disodium edetate (0.1% w/v), 2-hydroxypropyl-γ-cyclodextrin (2% w/v), propylene glycol (2% w/v), trometamol, tyloxapol and hydrochloric acid in purified water. According to WO1997/010805A1 (Novartis AG) the antimicrobial preservation of the eye drops is obtained by combining propylene glycol and benzalkonium chloride.

The composition of these two marketed cyclodextrin-containing eye drops indicate that as per the state of the art it is only possible to obtain acceptable preservation of aqueous cyclodextrin compositions by combining two or more antimicrobial preservatives. (Sodium mercurothiolate (synonym: thiomersal) is no longer allowed in ophthalmic products.)

Several patent applications or patents address the cyclodextrin inactivation of antimicrobial preservatives, either by development of novel hydrophilic preservatives or by combination of two or more preservatives to obtain acceptable efficacy.

JP60149530A (Takeda Chem. Ind.) discloses cyclodextrin-containing water-based pharmaceutical preparations containing a chlorhexidine derivative as an antimicrobial preservative.

WO1997010805A1 (Novartis AG) discloses a preserved ophthalmic composition comprising a cyclodextrin, a quaternary ammonium salt such as benzalkonium chloride, an alkylene glycol such as propylene glycol and a pharmaceutically active compound. According to the application, the amount of a cyclodextrin used may range from 0.01 to as high as 20% (w/w) and the active antimicrobial preservative consists of a combination of a quaternary ammonium salt and an alkylene glycol such as combination of 2.0% (w/v) propylene glycol and 0.01% (w/v) benzalkonium chloride.

EP 0 877 600 B1 (Alcon Manufacturing Ltd) discloses antimicrobial preservation of aqueous pharmaceutical compositions containing cyclodextrins. The patent describes that aqueous pharmaceutical compositions containing a pharmaceutically active compound and a cyclodextrin can be preserved using an antimicrobial preservative system comprising a combination of boric acid and one or more compounds selected from the group consisting of C16 benzalkonium halide compounds, polymeric quaternary ammonium compounds, and quaternary ammonium alkylene glycol phospholipid derivatives. Thus, a combination of boric acid and one or more quaternary ammonium preservatives are used to obtain acceptable antimicrobial preservation.

US 6,969,706 B1 (Allergan Inc.) discloses a composition comprising a cyclodextrin, guanidine-based cationic compound, and sorbic acid or sorbate. According to the claims the concentration of sorbic acid (or sorbate) can be from 0.05% to 1% and the pH from 4 to 6. An efficacy test (USP, Ph Eur-A, and Ph Eur-B Antimicrobial Preservative Efficacy Test) showed that the combination of polyhexamethylene biguanide and sorbate are effective against all of the tested pathogens in all of the tests when the concentration of polyhexamethylene biguanide is 3 ppm or greater.

US 10,463,677 B2 (Cydex Pharmaceuticals Inc.) discloses ophthalmic compositions containing latanoprost, sulfobutylether-β-cyclodextrin and up to 0.2% (w/v) of a preservative such as potassium sorbate or sorbic acid, parabens or others. The patent states that the inclusion of a preservative in the composition is optional, since the formulation is self-preserved by sulfobutylether-β-cyclodextrin depending on its concentration in solution.

Thus, the prior art indicates that in general two or more preservatives are necessary to obtain acceptable preservation of aqueous cyclodextrin formulation, especially with regard to higher cyclodextrin concentrations and natural cyclodextrins.

If aqueous eye drops contained in conventional ophthalmic multidose containers do not per se have adequate antimicrobial activity, antimicrobial preservative agents must be added to prevent microbial contamination that can occur during normal storage and use, and present hazard to the patient from infection and spoilage of the eye drops. An ideal antimicrobial preservative agent should be compatible with the active pharmaceutical ingredient, have no pharmacological and toxicological effect, have a broad antimicrobial effect, be water-soluble, have no effect on the pH of the aqueous eye drop media, be non-toxic and non-irritating, be chemically stable, tolerate autoclaving, have no effect on the eye drop container, and be effective at relatively low concentrations (E. M. Messmer, Konservierungsmittel in der Ophthalmologie, Ophthalmologe, 109, 1064-1070, 2012). In general, ophthalmic antimicrobial preservatives are not able to fulfill all these requirements and most cause some side effects at the ocular surface. The efficacy of an antimicrobial preservative may be diminished by the mode of formulation and by the excipients used to formulate the active ingredient in the aqueous eye drop medium. For example, in oil-in-water emulsions lipophilic antimicrobial preservatives are distributed between the oil and the aqueous phase and only the fraction in the aqueous phase will affect the microorganism (Alexander T. Florence and David Attwood, Physiochemical principles of pharmacy, 4^{th} Ed., Chapter 7.3.7 Preservative availability in emulsified systems, pages 249-250, Pharmaceutical Press, London 2006). Likewise, adsorption of preservatives to surfaces of glass and plastic containers, as well as to surfaces of particles in pharmaceutical microsuspensions, will reduce their efficacy. During eye drop formulation development the efficacy of the antimicrobial preservation must be tested in the final formulation and packaging to show that it fulfills the requirements of the appropriate targeted regulatory standard like either the European Pharmacopoeia (5.1.3. Efficacy of antimicrobial preservation; European Pharmacopoeia 10.5, 2021) or the USP-NF (Antimicrobial Effectiveness Testing <51>; United States Pharmacopeia, 2021).

A wide variety of antimicrobial preservatives exist but only a small fraction of them is used in eye drops. In principle, preservatives can be divided into three categories, a) detergent preservatives, b) oxidizing preservatives, and c) ionic-buffer preservatives (P. David Freeman and Malik Y. Kahook, Preservatives in topical ophthalmic medications: historical and clinical perspectives, Expert Rev. Ophthalmol. 4 (1), 59-64, 2009).

Detergent preservatives cause bacterial cell death by disrupting cell membranes with consecutive cell lysis. Examples of detergent preservatives are quaternary ammonium compounds like benzalkonium chloride (BAC), which is the most common antimicrobial preservative in ophthalmic products, cetrimonium chloride, benzethonium chloride, and chlorobutanol. Polyquaternium-1 (PolyQuad®) is like BAC a quaternary ammonium compound and regarded as detergent preservative although the molecule lacks a hydrophobic domain. Also, parabens (parahydroxybenzoate esters) are regarded as detergent preservatives although their mode of action is somewhat different. Oxidizing preservatives penetrate into the bacterial cell and affect the cellular metabolism. Examples of oxidizing preservatives include sodium perborate (GenAqua™), and stabilized oxychloro complex (Purite®, Bio-Cide International Inc., USA). The oxychloro complex dissociates to form chloride, oxygen, chlorine, chlorite and sodium salts when it comes into contact with light.

Sorbic acid is sometimes referred to as oxidizing preservative although its mode of action is different (Fernando Aguilar, Riccardo Crebelli, Alessandro Di Domenico, Birgit Dusemund, Maria Jose Frutos, Pierre Galtier, David Gott, Ursula Gundert-Remy, Claude Lambré, Jean-Charles Leblanc, Oliver Lindtner, Peter Moldeus, Alicja Mortensen, Pasquale Mosesso, Dominique Parent-Massin, Agneta Oskarsson, Ivan Stankovic, Ine Waalkens-Berendsen, Rudolf Antonius Woutersen, Matthew Wright and Maged Younes, Scientific opinion on the re-evaluation of sorbic acid (E 200), potassium sorbate (E 202) and calcium sorbate (E 203) as food additives, EFSA Journal, 13 (6), 1-91, 2015).

The bactericidal effect of the ionic-buffer preservatives is also based on oxidative mechanism. An example of an ionic-buffer preservative is SofZia® (Alcon, USA) that consists of a combination of boric acid, zinc, sorbitol and propylene glycol.

### SUMMARY

Conventional containers that contain multiple doses of drug formulation (in short: multidose compositions) are exposed to the atmosphere and its pathogens several times over several days. Thus, it is usually necessary to include preservatives in multidose compositions.

The preservation of multidose compositions is facing the challenge that only a limited number of compounds have obtained regulatory approval so far, a minimum yet effective concentration of preservative is desired to be included, and preferably only one preservative shall be included to simplify formulation procedure and reduce or even avoid incompatibilities with other excipients.

In addition, while developing formulations with cyclodextrins, the inventors noticed that typical preservatives such as benzalkonium chloride were not efficient enough at recommended concentrations for ophthalmic use, in particular as preservatives for multidose compositions.

The present disclosure provides well-preserved aqueous cyclodextrin multidose compositions which are advantageous in that they require the addition of only one antimicrobial preservative at concentrations which have obtained regulatory approval and even in some embodiments rather low concentrations, and which are customary in the art and products sold in the market. In particular, the inclusion of other preservatives, such as benzalkonium chloride could be avoided.

In one aspect the present disclosure provides an aqueous composition comprising
drug/cyclodextrin complexes which comprise a drug and an alpha-, beta- or gamma-cyclodextrin; and
a preservative which comprises or essentially consists of sorbic acid or a pharmaceutically acceptable sorbic acid salt.

In another aspect the present disclosure provides an aqueous composition comprising
drug/cyclodextrin complexes which comprise a drug and a gamma-cyclodextrin; and
a preservative which comprises or essentially consists of sorbic acid or a pharmaceutically acceptable sorbic acid salt.

In another aspect the present disclosure provides a method for treating a condition of the posterior segment and/or the anterior segment of the eye in a subject in need thereof, said method comprising applying topically to the eye surface of said subject, an aqueous composition according to any one of claims 1 to 23, comprising a drug as the active principle, in an amount which delivers a therapeutically effective amount of said drug to said segment or segments of the eye.

In another aspect the present disclosure provides a method for avoiding bacterial contamination of an eye drop formulation comprising cyclodextrin, said method comprising adding a preservative efficient amount of sorbic acid or its pharmaceutically acceptable salts in said eye drop formulation.

In a further aspect the present disclosure provides a process for manufacturing an eye drop formulation comprising cyclodextrin, said process comprising
(i) providing an eye drop formulation comprising drug/cyclodextrin complexes which comprise a drug and an alpha-, beta- or gamma-cyclodextrin; and
(ii) adding a preservative efficient amount of sorbic acid or its pharmaceutically acceptable salts in said eye drop formulation.

### DETAILED DESCRIPTION

Further aspects, features and advantages of the exemplary embodiments will become apparent from the detailed description which follows.

The patents, published applications and scientific literature referred to herein establish the knowledge of those with skill in the art and are hereby incorporated by reference in their entireties to the same extent as if each was specifically and individually indicated to be incorporated by reference.

As used herein, whether in a transitional phrase or in the body of a claim, the terms “comprise(s)” and “comprising” are to be interpreted as having an open-ended meaning. That is, the terms are to be interpreted synonymously with the phrases “having at least” or “including at least”. When used in the context of a method, the term “comprising” means that the method includes at least the recited steps, but may include additional steps. When used in the context of a composition, the term “comprising” means that the composition includes at least the recited features or components, but may also include additional features or components.

The terms “consists essentially of” or “consisting essentially of” have a partially closed meaning, that is, they do not permit inclusion of steps or features or components which would substantially change the essential characteristics of a method or composition; for example, steps or features or components which would significantly interfere with the desired properties of the compounds or compositions described herein, i.e., the method or composition is limited to the specified steps or materials and those which do not materially affect the basic and novel characteristics of the method or composition. The terms “consists of” and “consists” are closed terminology and allow only for the inclusion of the recited steps or features or components.

As used herein, the singular forms “a,” “an” and “the” specifically also encompass the plural forms of the terms to which they refer, unless the content clearly dictates otherwise.

The term “about” is used herein to mean approximately, in the region of, roughly, or around. When the term “about” is used in conjunction with a numerical range, it modifies that range by extending the boundaries above and below the numerical values set forth. In general, the term “about” or “approximately” is used herein to modify a numerical value above and below the stated value by a variance of 10%.

The term “dissolved” or “substantially dissolved” is used herein to mean the solubilization of a solid in a solution. It can be considered that a solid is “dissolved” or “substantially dissolved” in a solution when the resulting solution is clear or substantially clear.

As used herein, the recitation of a numerical range for a variable is intended to convey that the variable can be equal to any values within that range. Thus, for a variable which is inherently discrete, the variable can be equal to any integer value of the numerical range, including the end-points of the range. Similarly, for a variable which is inherently continuous, the variable can be equal to any real value of the numerical range, including the end-points of the range. As an example, a variable which is described as having values between 0 and 2, can be 0, 1 or 2 for variables which are inherently discrete, and can be 0.0, 0.1, 0.01, 0.001, or any other real value for variables which are inherently continuous.

In the specification and claims, the singular forms include plural referents unless the context clearly dictates otherwise. As used herein, unless specifically indicated otherwise, the word “or” is used in the “inclusive” sense of “and/or” and not the “exclusive” sense of “either/or.”

Technical and scientific terms used herein have the meaning commonly understood by one of skill in the art to which the present description pertains, unless otherwise defined. Reference is made herein to various methodologies and materials known to those of skill in the art. Standard reference works setting forth the general principles of pharmacology and pharmaceutics include Goodman and Gilman's The Pharmacological Basis of Therapeutics, 10^{th} Ed., McGraw Hill Companies Inc., New York (2001) and Remington, The Science and Practice of Pharmacy, 22^{nd} Ed., Philadelphia (2013).

As used herein the term “% by weight of a compound X based on the volume of the composition”, also abbreviated as “% w/v”, corresponds to the amount of compound X in grams that is introduced in 100 mL of the composition.

As used herein the term “microparticle” refers to a particle having a diameter D₅₀ of 1 μm or greater to about 200 μm. The term “nanoparticle” refers to a particle having a diameter D₅₀ of less than 1 μm.

As used herein an “ocular condition” is a disease, ailment or other condition which affects or involves the eye, one of the parts or regions of the eye, or the surrounding tissues such as the lacrimal glands. Broadly speaking, the eye includes the eyeball and the tissues and fluids which constitute the eyeball, the periocular muscles (such as the oblique and rectus muscles), the portion of the optic nerve which is within or adjacent to the eyeball and surrounding tissues such as the lacrimal glands and the eye lids.

As used herein an “anterior ocular condition” is a disease, ailment or condition which affects or which involves an anterior (i.e. front of the eye) ocular region or site, such as a periocular muscle, an eye lid, lacrimal gland or an eye ball tissue or fluid which is located anterior to the posterior wall of the lens capsule or ciliary muscles. Thus, an anterior ocular condition primarily affects or involves one or more of the following: the conjunctiva, the cornea, the anterior chamber, the iris, the lens, or the lens capsule, and blood vessels and nerves which vascularize or innervate an anterior ocular region or site. An anterior ocular condition is also considered herein as extending to the lacrimal apparatus. In particular, the lacrimal glands which secrete tears, and their excretory ducts which convey tear fluid to the surface of the eye. Furthermore, this includes neovascularization of the cornea, including corneal neovascularization associated with corneal inflammation, including herpes simplex keratitis, herpes zoster keratitis, bacterial corneal infections, fungal corneal infections and corneal graft rejection. It also includes iris neovascularization and neovascular glaucoma, which may be associated with retinal vein occlusion, diabetic retinopathy, other ischemic retinopathies and carotid stenosis. It also includes inflammation following ocular surgery, typically cataract surgery. More broadly, “anterior ocular conditions” are those which affect also the external ocular surface. Moreover, an anterior ocular condition affects or involves the posterior chamber, which is the narrow space between the iris and lens.

A “posterior ocular condition” is a disease, ailment or condition which primarily affects or involves a posterior ocular region or site such as the retina or choroid (in a position posterior to a plane through the posterior wall of the lens capsule), vitreous, vitreous chamber, retina, optic nerve (i.e. the optic disc), and blood vessels and nerves which vascularize or innervate a posterior ocular region or site.

Thus, a posterior ocular condition can include a disease, ailment or condition such as, for example, macular degeneration (such as non-exudative age-related macular degeneration and exudative age-related macular degeneration, also known as wet or neovascular age related macular degeneration); choroidal neovascularization; pachychoroidal disorders; polypoidal choroidal vasculopathy; acute macular neuroretinopathy; macular edema (such as cystoid macular edema and diabetic macular edema); Behcet's disease, retinal disorders, diabetic retinopathy (including proliferative diabetic retinopathy and diabetic macular edema; also non-proliferative diabetic retinopathy); retinal arterial occlusive disease; central retinal vein occlusion; branch retinal vein occlusion; sickle cell retinopathy; uveitic retinal disease also known as posterior uveitis, including macular edema associated with inflammation and neovascularization associated with inflammation; sarcoidosis retinal inflammation; sarcoidosis uveitis; syphilitic uveitis; systemic lupus erythematosus related inflammation in retina or retinal vessels; retinal detachment; proliferative vitreoretinopathy; ocular trauma which affects a posterior ocular site or location; a posterior ocular condition caused by or influenced by an ocular laser treatment; posterior ocular conditions caused by or influenced by a photodynamic therapy; photocoagulation; radiation retinopathy; epiretinal membrane disorders; branch retinal vein occlusion; anterior ischemic optic neuropathy.

One aspect of the present description concerns ophthalmic compositions for topical drug delivery to the eye(s) and to methods for the treatment of a posterior ocular condition. In preferred embodiments, the ophthalmic compositions are used for the treatment of pathological states that arise or are exacerbated by ocular angiogenesis and vascular leakage, for example, in diabetic retinopathy (including background diabetic retinopathy, proliferative diabetic retinopathy and diabetic macular edema); age-related macular degeneration (AMD) (including neovascular (wet/exudative) AMD, dry AMD, and Geographic Atrophy); pathologic choroidal neo vascularization (CNV) from any mechanism (i.e. high myopia, trauma, sickle cell disease; ocular histoplasmosis, angioid streaks, traumatic choroidal rupture, drusen of the optic nerve, and some retinal dystrophies); pathologic retinal neovascularization from any mechanism (i.e., sickle cell retinopathy, retinopathy of prematurity, Eales disease, ocular ischemic syndrome, carotid cavernous fistula, familial exudative vitreoretinopa thy, hyperviscosity syndrome, idiopathic occlusive arteriolitis, birdshot retinochoroidopathy, retinal vasculitis, sarcoidosis, or toxoplasmosis); uveitis; retinal vein occlusion (central or branch); ocular trauma; surgery induced edema; surgery induced neovascularization; cystoid macular edema; ocular ischemia; retinopathy of prematurity; Coats disease; sickle cell retinopathy and/or neovascular glaucoma.

In another aspect, the ophthalmic compositions may be used for the treatment of anterior ocular conditions including inflammation following ocular surgery, typically following cataract surgery.

As per the instant disclosure, the aqueous composition is an ophthalmically acceptable medium. The term “ophthalmically acceptable medium” is intended to mean a medium suitable for topical ophthalmic administration of the composition, for example, in the form of aqueous eye drop microsuspension containing at least 60% (w/v) purified water. In a specific embodiment, the aqueous composition is an unbuffered aqueous eye drop vehicle. In another specific embodiment the aqueous composition can be a true solution. In specific preferred embodiments, the aqueous composition is a multidose composition.

### Sorbic Acid

The compositions of the present disclosure comprise sorbic acid and its salts such as potassium sorbate, sodium sorbate and calcium sorbate. The chemical name of sorbic acid is (2E,4E)-hexa-2,4-dienoic acid (molecular weight: 112.12 g/mol) and synonyms include trans, trans-2,4-hexadienoic acid, 2,4-hexadienoic acid (E,E), and (E,E)-1,3-pentadiene-1-carboxylic acid. Sorbic acid is a white free-flowing powder. It is somewhat lipophilic (Log P_{(octanol/water)}=1.3) slightly soluble in water and soluble in ethanol. The melting point of sorbic acid is between 133 and 135° C. and its pKa 4.76. Monographs of sorbic acid and potassium sorbate materials that can be used in pharmaceutical drug compositions are included in both the European Pharmacopoeia 10.7 and the United States Pharmacopeia/National Formulary (USP43-NF38).

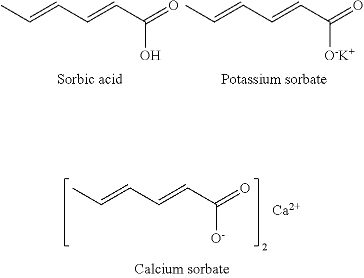

Sorbates inhibit microbial growth by inducing alterations in the morphology, integrity and function of cell membranes and by inhibiting transport functions and metabolic activity. Sorbates are also known to inhibit the in vitro activity of many enzymes, especially sulfhydryl-containing enzymes. Other mechanisms of action have also been proposed (Fernando Aguilar, Riccardo Crebelli, Alessandro Di Domenico, Birgit Dusemund, Maria Jose Frutos, Pierre Galtier, David Gott, Ursula Gundert-Remy, Claude Lambré, Jean-Charles Leblanc, Oliver Lindtner, Peter Moldeus, Alicja Mortensen, Pasquale Mosesso, Dominique Parent-Massin, Agneta Oskarsson, Ivan Stankovic, Ine Waalkens-Berendsen, Rudolf Antonius Woutersen, Matthew Wright and Maged Younes, Scientific opinion on the re-evaluation of sorbic acid (E 200), potassium sorbate (E 202) and calcium sorbate (E 203) as food additives, EFSA Journal, 13 (6), 1-91, 2015).

In specific embodiments, the aqueous compositions of the present disclosure comprise only sorbic acid or pharmaceutically acceptable sorbate salts as antimicrobial preservatives. No additional preservatives are added. Thus, the preservative that is used in the aqueous compositions of the present disclosure consists essentially of sorbic acid or sorbate salts. In some examples the preservative consists of sorbic acid or sorbate salts.

In specific embodiments, the antimicrobial preservative agent does not comprise a conventional topical ophthalmic antimicrobial compound selected from the group consisting of benzalkonium chloride, benzethonium chloride, butyl-para-hydroxybenzoate, chlorobutanol, methyl-para-hydroxybenzoate, polyguaternium-1, propyl-para-hydroxybenzoate, or an oxidizing antimicrobial preservative agent including perborate, zinc or chlorite ions.

### Cyclodextrins

Cyclodextrins are cyclic oligosaccharides consisting of (α-1,4)-linked D-glucopyranose units. The most common natural cyclodextrins, and the only natural ones used in pharmaceutical products, are α-cyclodextrin (alpha-cyclodextrin), β-cyclodextrin (beta-cyclodextrin) and γ-cyclodextrin (gamma-cyclodextrin) consisting of 6, 7 and 8 D-glucopyranose units. Cyclodextrins are doughnut-shaped molecules with a hydrophilic outer surface and a somewhat lipophilic central cavity.

In aqueous solutions cyclodextrins are able to form water-soluble inclusion complexes with lipophilic poorly-soluble drugs by taking up some lipophilic moiety of the drugs into the central cavity (P. Jansook, N. Ogawa and T. Loftsson, 2018. Cyclodextrins: structure, physicochemical properties and pharmaceutical applications. International Journal of Pharmaceutics 535, 272-284). In aqueous solutions drug/cyclodextrin complexes are in dynamic equilibrium with dissolved drug and cyclodextrin molecules. The rates for formation and dissociation of drug/cyclodextrin complexes are very close to the diffusion-controlled limits and in aqueous solutions drug/cyclodextrin complexes are continuously being formed and dissociated (Marcus E. Brewster, Thorsteinn Loftsson, 2007. Cyclodextrins as pharmaceutical solubilizers, Advanced Drug Delivery Reviews, 59 (7), 645-66; V. J. Stella, V. M. Rao, E. A. Zannou, V. Zia, 1999. Mechanisms of drug release from cyclodextrin complexes, Advanced Drug Delivery Reviews, 36 (1), 3-16).

Due to their cyclic structure and intramolecular hydrogen bond formations, the three natural cyclodextrins and their complexes are much less soluble in aqueous solutions than linear oligosaccharides of comparable molecular weight. Like starch, cyclodextrins can be reacted with a wide variety of reagents to obtain water-soluble cyclodextrin derivatives. For example, hydroxypropylated cyclodextrin derivatives, such as 2-hydroxypropyl-β-cyclodextrin and 2-hydroxypropyl-γ-cyclodextrin, are obtained by treating the natural cyclodextrins with propylene oxide, sulfobutylether β-cyclodextrin and sulfobutylether γ-cyclodextrin by treating the natural cyclodextrins with 4-butane sultone, and randomly methylated cyclodextrins, such as randomly methylated β-cyclodextrin, are obtained by treating the natural cyclodextrins with methyl iodide.

In specific embodiments, the compositions of the present disclosure comprise α-cyclodextrin, β-cyclodextrin or γ-cyclodextrin consisting of 6, 7 and 8 D-glucopyranose units. In specific embodiments, the compositions preferably comprise γ-cyclodextrin consisting of 8 D-glucopyranose units.

The present disclosure more specifically concerns compositions with natural γ-cyclodextrin and comprising nano- and (preferably) microparticles composed of drug/γ-cyclodextrin complexes (P. Saokham, T. Loftsson, γ-Cyclodextrin, International Journal of Pharmaceutics, 517, 278-292, 2017).

Typically, the amount of cyclodextrin in the aqueous compositions may be from 1 to 35%, in particular 5 to 30%, more particularly 10 to 25%, by weight of cyclodextrin based on the volume of the composition.

Now it has been surprisingly discovered that aqueous cyclodextrin formulations containing α-cyclodextrin, β-cyclodextrin and γ-cyclodextrin, and in particular the natural γ-cyclodextrin, at relatively high concentrations can be preserved by including sorbic acid or sorbate salts at concentrations not exceeding those found in currently marketed aqueous eye drops without necessity to include additional antimicrobial preservatives. The aqueous compositions of the present disclosure may comprise only sorbic acid or sorbate salts as preservatives. In specific embodiments, no additional antimicrobial preservatives are added. Thus, in specific embodiments, the preservative that is used in the aqueous compositions consists essentially of sorbic acid or sorbate salts. In some examples, the preservative consists of sorbic acid or sorbate salts. Sorbic acid and sorbate salts do possess antioxidative activity and can, under certain conditions stabilize the pH of aqueous drug formulations, such as aqueous corticosteroid eye drop suspensions. More specifically, sorbic acid and sorbate salts can stabilize the pH of aqueous eye drop suspensions comprising solid complexes of dexamethasone and gamma-cyclodextrin.

In specific embodiments, the composition does not comprise a conventional topical ophthalmic antimicrobial agent selected from the group consisting of benzalkonium chloride, benzethonium chloride, butyl-para-hydroxybenzoate, chlorobutanol, methyl-para-hydroxybenzoate, polyguaternium-1, propyl-para-hydroxybenzoate, or oxidizing antimicrobial preservative agents including perborate, zinc or chlorite ions

### Drug

The aqueous composition of the disclosure comprises a drug and can be for either human or veterinary use. In the context of the disclosure, the drug is preferably an ophthalmic drug, that is a compound that exhibits a therapeutic effect when topically administered on the eye surface in a sufficient amount to a patient suffering from an ocular condition. The antimicrobial preservative composition disclosed in the present invention is also suitable for any other aqueous formulations to be leveraged for topical use, such as in dermatology (for skin and nails), otorhinology and laryngology (for ear, nose and mouth), proctology and gynecology.

Examples of ocular conditions which may be treated by the compositions and methods of the invention include, but are not limited to, uveitis, macular edema, macular degeneration, retinal detachment, ocular tumors, fungal or viral infections, multifocal choroiditis, diabetic retinopathy, proliferative vitreoretinopathy (PVR), sympathetic ophthalmia, Vogt Koyanagi-Harada (VKH) syndrome, histoplasmosis, uveal diffusion, and vascular occlusion.

The active agent for use in the ophthalmic compositions of the invention can be selected from the group consisting of angiotensin-converting enzyme inhibitors (ace-inhibitors), kinase inhibitors, agents that influence basement membrane, agents that influence the growth of endothelial cells, adrenergic agonists or blockers, cholinergic agonists or blockers, aldose reductase inhibitors, analgesics, anesthetics, antiallergics, anti-inflammatory agents, steroids (such as steroidal anti-inflammatory agents), antihypertensives, pressors, antibacterials, antivirals, antifungals, antiprotozoals, anti-glaucoma agents, anti-infective agents, antitumor agents, antimetabolites, and antiangiogenic agents. Other drugs include chloride channel activators (against dry eye disease), small-molecule inhibitors of the NLRP3 inflammasome (e.g., MCC-950) for the treatment of inflammatory diseases, ecothiopathe iodine (a cholinesterase inhibitor against glaucoma), and atropine (for stimulation conjunctival goblet cells and mucin formation in conjunctiva).

The steroidal anti-inflammatory agents that may be used in the ocular compositions and methods of the invention include, but are not limited to, 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, etiprednol dicloacetate, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, triamcinolone hexacetonide, and any of their derivatives having anti-inflammatory activity. Nonsteroidal anti-inflammatory agents that may be used include, but are not limited to, aspirin, bromfenac, diclofenac, flurbiprofen, ibuprofen, indomethacin, ketorolac, naproxen, nepafenac, and suprofen.

The ACE-inhibitors (i.e., angiotensin-converting enzyme inhibitors drugs) that may be used in the ocular compositions and methods of the invention include, but are not limited to, benazepril, captopril, enalapril, fosinopril, lisinopril, moexipril, perindopril, quinapril, ramipril and trandolapril.

The kinase inhibitors (anti-VEGF) that may be used in the ocular compositions and methods of the invention include, but are not limited to, acrizanib, afatinib, alectinib, altiratinib, anlotinib, apatinib, avapritinib, axitinib, bosutinib, brivanib, cabozantinib, calabrutinib, cediranib, crizotinib, dacomitinib, dasatinib, dovitinib, entrectinib, erlotinib, foretinib, fruquintinib, golvatinib, gilteritinib, ibrutinib, imatinib, lapatinib, lenvatinib, linifanib, midostaurin, motesanib, neratinib, nilotinib, nintedanib, orantinib, pacritinib, pazopanib, pexidartinib, ponatinib, quizartinib, regorafenib, semaxatinib, sitravatinib, sorafenib, sunitinib, surufatinib, telatinib, tivozanib, tucatinib, vandetanib, vatalanib, zanubrutinib, and ziv-aflibercept, as well as 2-D08, AZD2932, BAW2881, BFH772, BMS-794833, CYC116, Ki8751, LY2874455, MGCD-265 and its analogues, OSI-930, PD173074, RAF-265, SU5402, and ZM 323881.

Vanilloid receptor (TRPV1) antagonists that may be used in the ocular compositions and methods of the invention include, but are not limited to PHE-377, JNJ-38893777, MR-1817, XEN-D0501, thiourea analogues such as capsazepine, urea analogues such as A-425619, GRC-6211, V116517, SB-705498 and JNJ-17203212, cinnamide analogues such as SB-366791, asivatrep (PAC-14028) and AMG-9810, quinazolinone analogues such as AMG-517, SAF312 (libvatrep) and MK-2295, benzimidazole analogues such as ABT-102, AMG-2674, A-784168, mavatrep (JNJ-39439335), DWP05195 and ACD440, and imidazolidine analogues such as AZD-1386.

Angiotensin II receptor antagonists (i.e., sartans) that may be used in the ocular compositions and methods of the invention include, but are not limited to, azilsartan, candesartan, eprosartan, irbesartan, losartan, olmesartan, telmisartan, and valsartan.

Carbonic anhydrase inhibitors that may be used in the ocular compositions and methods of the invention include, but are not limited to, acetazolamide, brinzolamide, dorzolamide, ethoxzolamide, and methazolamide.

The antiglaucoma and ocular antihypertension agents that may be used in the ocular compositions and methods of the invention include, but are not limited to, prostaglandin analogs such as bimatoprost, latanoprost, travoprost, tafluprost, latanoprostene-bunod, and NCX-470, β-blockers such as carteolol, timolol, betaxolol, and levobunolol, and carbonic anhydrase inhibitors such as acetazolamide, metipranolol, methazolamide, dorzolamide, brinzolamide, diclofenamide, ethoxzolamide, and zonisamide, or any combinations thereof.

Antibacterial agents that may be used in the ocular compositions and methods of the invention include, but are not limited to, tigecycline, ofloxacin, metronidazole, clarithromycin, clindamycin, chloramphenicol, rifampicin, azithromycin and isoniazid.

Antifungal agents that may be used in the ocular compositions and methods of the invention include, but are not limited to, isavuconazole, voriconazole, and caspofungin.

Antiviral agents that may be used in the ocular compositions and methods of the invention include, but are not limited to, zidovudine, sorvudine, famiclovir, vabaciclovir, acyclovir, ganciclovir, rimantadine, amprenavir, atazanavir, indinavir, nelfinavir, saquinavir, tipranavir, abacavir, and nevirapine.

Immunomodulators agents like ciclosporin, tacrolimus, everolimus, pimecrolimus, temsirolimus and sirolimus.

In preferred embodiments, the aqueous compositions of the disclosure comprise drugs, preferably drugs that are poorly soluble in water, complexed with cyclodextrin which may advantageously be used as topical formulation for treating posterior ocular conditions.

In preferred embodiments, the aqueous composition of the disclosure comprises a steroid, preferably dexamethasone.

The amount of active pharmaceutical ingredient in the composition may be 0.001% (w/v) to 10% (w/v), preferably 0.1% (w/v) to 5% (w/v), more preferably 0.5% (w/v) to 4% (w/v), even more preferably particularly 1% (w/v) to 3% (w/v) by weight of the active pharmaceutical ingredient based on the volume of the composition. In specific embodiments, the concentration of dexamethasone may be 0.001% (w/v) to 10% (w/v), preferably 0.1% (w/v) to 5% (w/v), more preferably 0.5% (w/v) to 4% (w/v), even more preferably 1% (w/v) to 3% (w/v) by weight of the active pharmaceutical ingredient based on the volume of the composition.

### Drug/Cyclodextrin Microsuspension

In specific embodiments, the aqueous composition is an aqueous microsuspension, comprising microparticles of solid drug/cyclodextrin complexes, suspended in an aqueous medium. As used herein the term “microparticle” refers to a particle having a diameter D₅₀ of about 1 μm to about 200 μm. The term “nanoparticle” refers to a particle having a diameter D₅₀ of less than 1 μm. In exemplary embodiments, the diameter D₅₀ of microparticles is 1 μm or greater to about 200 μm, in particular 1 μm to about 100 μm; and the nanoparticles have a D₅₀ of less than about 1 μm.

In particular, the aqueous composition of the disclosure is a microsuspension comprising solid aggregates of drug/cyclodextrin complexes that have a diameter D₅₀ of less than about 100 μm, in particular about 1 μm to about 100 μm. In one embodiment, the diameter D₅₀ may be in the range of about 1 μm to about 50 μm, in particular about 1 μm to about 30 μm, more particularly about 1 μm to about 25 μm.

As per the present disclosure, the diameter D₅₀ is measured by laser diffraction particle size analysis. Generally, there are a limited number of techniques for measuring/evaluating cyclodextrin/drug particle or complex diameter and/or size. In particular, persons of ordinary skill in this field know that the physical properties (e.g. particle size, diameter, average diameter, mean particle size, etc.) are typically evaluated/measured using such limited, typical known techniques. For example, such known techniques are described in Int. J. Pharm. 493 (2015), 86-95, which is incorporated by reference herein in its entirety. In addition, such limited, known measurement/evaluation techniques were known in the art as evidenced by other technical references such as, for example, European Pharmacopoeia (2.9.31 Particle size analysis by laser diffraction, January 2010), and Saurabh Bhatia, Nanoparticles types, classification, characterization, fabrication methods and drug delivery applications, Chapter 2, Natural Polymer Drug Delivery Systems, PP. 33-94, Springer, 2016, which are also incorporated by reference herein in their entireties.

Preferred methods for making microsuspension with solid aggregates of drug/cyclodextrin complexes are for example described in WO2018/100434 which content is incorporated herein in its entirety.

In particular, 60 to 95% by weight, more particularly 70 to 90% by weight, of the drug in the composition may be in the form of a solid complex of drug and cyclodextrin. Even more particularly, 5 to 40% by weight, in particular 10 to 30% by weight, of the drug in the composition may be in dissolved form. The dissolved form includes uncomplexed drug that is dissolved in the liquid phase and complexes of drug and cyclodextrin that are dissolved in the liquid phase as well as water-soluble nanoparticles consisting of drug/cyclodextrin complex aggregates.

Preferably, 0% to 0.5% by weight of the drug in the composition may be in uncomplexed solid form. As such, the composition of the disclosure may be substantially free of solid uncomplexed particles of drug.

In one embodiment, the microsuspension may comprise about 70% to about 99% of the drug in microparticles and about 1% to about 30% of the drug in nanoparticles. More particularly, the microsuspension may comprise about 80% to about 95% of the drug in microparticles having a diameter of about 1 μm to about 100 μm, and about 20% to about 5% of the drug in nanoparticles. The microsuspension may comprise about 80% of the drug in microparticles having a diameter of about 1 μm to about 100 μm, and about 20% of the drug in nanoparticles.

In another embodiment, the microsuspension may comprise about 40% to about 99% of the drug in microparticles and about 1% to about 60% of the drug in nanoparticles or water-soluble drug/cyclodextrin complexes. In particular, the microsuspension may comprise about 80% to about 95% of the drug in microparticles having a diameter of about 1 μm to about 100 μm, and about 5% to about 20% of the drug in nanoparticles or water-soluble active pharmaceutical ingredient/cyclodextrin complexes.

According to a preferred embodiment, the aqueous compositions of the present disclosure are a microsuspension comprising solid aggregates of drug/cyclodextrin complexes, preferably corticosteroid/cyclodextrin complexes, and more preferably dexamethasone/γ-cyclodextrin complexes.

The aqueous compositions of the present disclosure may further comprise a polymer.

In particular, said polymer may be a water-soluble polymer. Moreover, said polymer may be a surface active polymer. The term “surface active polymer” is intended to mean a polymer that exhibits surfactant properties enabling to reduce the whole surface tension of the formulation. The polymer enhances the physical stability of the composition. Surface active polymers may, for example, comprise hydrophobic chains grafted to a hydrophilic backbone polymer; hydrophilic chains grafted to a hydrophobic backbone; or alternating hydrophilic and hydrophobic segments. The first two types are called graft copolymers and the third type is named block copolymer.

In one embodiment, the composition comprises a polymer selected from the group consisting of a polyoxyethylene fatty acid ester; a polyoxyethylene alkylphenyl ether; a polyoxyethylene alkyl ether; a cellulose derivative such as alkyl cellulose, hydroxyalkyl cellulose and hydroxyalkyl alkylcellulose; a carboxyvinyl polymer such as a carbomer and polycarbophil, for example Carbopol® 971, Carbopol® 974 and Noveon® AA1; a polyvinyl polymer; a polyvinyl alcohol; a polyvinylpyrrolidone; a copolymer of polyoxypropylene and polyoxyethylene; tyloxapol; and combinations thereof.

Other polymers possessing viscosity enhacing activity like hyaluronic acid or sodium hyaluronate (natural of reticulated), chitosan, xanthan gum, gellan gum, sodium alginate or iota-carrageenan.

Examples of suitable polymers include, but are not limited to, polyethylene glycol monostearate, polyethylene glycol distearate, hydroxypropyl methylcellulose, hydroxypropyl cellulose, polyvinylpyrrolidone, polyvinyl alcohol, polyoxyethylene lauryl ether, polyoxyethylene octyldodecyl ether, polyoxyethylene stearyl ether, polyoxyl hydroxy stearate (Kolliphor® HS15, from BASF), polyoxyethylene myristyl ether, polyoxyethylene oleyl ether, sorbitan esters, polyoxyethylene hexadecyl ether (e.g., cetomacrogol 1000), polyoxyethylene castor oil derivatives (Kolliphor® EL or Kolliphor® RH40 from BASF), polyoxyethylene sorbitan fatty acid esters (e.g., Tween® 20 and Tween® 80 (ICI Specialty Chemicals)); polyethylene glycols (e.g., Carbowax® 3550 and 934 (Union Carbide)), polyoxyethylene stearates, carboxymethylcellulose calcium, carboxymethylcellulose sodium, methylcellulose, hydroxyethylcellulose, hydroxypropyl methylcellulose, cellulose, polyvinyl alcohol (PVA), poloxamers (e.g., Pluronics® F68 and FI08, which are block copolymers of ethylene oxide and propylene oxide); poloxamines (e.g., Tetronic® 908, also known as Poloxamine 908, which is a tetrafunctional block copolymer derived from sequential addition of propylene oxide and ethylene oxide to ethylenediamine (BASF Wyandotte Corporation, Parsippany, N.J.)); Tetronic® 1508 (T-1508) (BASF Wyandotte Corporation), Tritons X-200, which is an alkyl aryl polyether sulfonate (Rohm and Haas); PEG-derivatized phospholipid, PEG-derivatized cholesterol, PEG-derivatized cholesterol derivative, PEG-derivatized vitamin A, PEG-derivatized vitamin E, random copolymers of vinyl pyrrolidone and vinyl acetate, combinations thereof and the like.

Tyloxapol is a 4-(1,1,3,3-tetramethylbutyl) phenol polymer with formaldehyde and oxirane.

More particularly, the copolymer of polyoxypropylene and polyoxyethylene may be a triblock copolymer comprising a hydrophilic block (polyoxyethylene)-hydrophobic block (polyoxypropylene)-hydrophilic block (polyoxyethylene) configuration, also named poloxamer.

In one embodiment, the composition of the disclosure comprises a polymer which is a poloxamer. Poloxamers can include any type of poloxamer known in the art. Poloxamers include poloxamer 101, poloxamer 105, poloxamer 108, poloxamer 122, poloxamer 123, poloxamer 124, poloxamer 181, poloxamer 182, poloxamer 183, poloxamer 184, poloxamer 185, poloxamer 188, poloxamer 212, poloxamer 215, poloxamer 217, poloxamer 231, poloxamer 234, poloxamer 235, poloxamer 237, poloxamer 238, poloxamer 282, poloxamer 284, poloxamer 288, poloxamer 331, poloxamer 333, poloxamer 334, poloxamer 335, poloxamer 338, poloxamer 401, poloxamer 402, poloxamer 403, poloxamer 407, poloxamer 105 benzoate and poloxamer 182 dibenzoate.

Especially useful polymers as stabilizers are poloxamers, preferably Poloxamer 407.

Preferred polymers are poloxamer, tyloxapol, polyalkylenglycol, hydroxyalkylcelluselose, hydroxyalkyl alkylcellylose, polyvinyl alcohol, polyoxyethylene ether, sodium carboxymethyl cellulose, polyethylene glycol, 12-hydroxystearate, polyoxyethylene sorbitan mono-oleate. Particularly preferred polymers are hydroxypropyl methylcellulose (HMPC), sodium carboxymethyl cellulose MW 250 kDa, and hydroxyethylcellulose.

The amount of polymer in the composition may be 0.1% (w/v) to 5% (w/v), in particular 0.3% (w/v) to 3% (w/v), more particularly 0.5% (w/v) to 2% (w/v) by weight of the polymer based on the volume of the composition.

### Antioxidants

The aqueous composition may comprise an additive to prevent the oxidation of the drug.

In a preferred embodiment, the additive to prevent the oxidation of the drug is selected from antioxidants, oxygen scavengers, chelating agents and mixtures thereof.

Antioxidants typically include phenolic antioxidants and reducing agents. Phenolic antioxidants are sterically hindered phenols that react with free radicals, blocking the oxidation reaction. Among phenolic antioxidants, one can cite butylated hydroxyanisole (BHA) and butylated hydroxytoluene (BHT). Reducing agents are compounds that have lower redox potential than the drug they are intended to prevent from oxidation. Reducing agents scavenge oxygen from the medium and thus delay or prevent oxidation. Among reducing agents, one can cite sodium thiosulfate (STS).

The phenolic antioxidants and reducing agents added to the composition to prevent drug or excipient oxidation can be added at a concentration of at least 0.05% (w/v), preferably at a concentration between 0.05% (w/v) and 1% (w/v), more preferably between 0.1 to 0.5%, 0.1 to 0.45%, 0.2 to 0.4%, 0.2 to 0.35 or 0.2 to 0.3, preferably 0.3 to 0.5 (w/v). In specific embodiments an antioxidant, for example sodium thiosulfate, may not be added to the compositions.

### Chelating Agents

Chelating agents are compounds that chelate metallic ions such as copper and iron that catalyze oxidation of the active pharmaceutical ingredient or excipients used to form the pharmaceutical product. Also, chelating agents are able to enhance the antioxidant action of tocopherols or phenolic compounds, resulting in synergistic antioxidant effect. Examples of chelating agents are divalent and polyvalent carboxylic acids and their salts. Preferred examples are ethylenediaminetetraacetic acid (EDTA), 2,2′,2″-nitrilotriacetic acid (NTA), iminodisuccinic acid (IDS), polyaspartic acid, S, S-ethylenediamine-N,N′-disuccinic acid (EDDS), methylglycinediacetic acid (MGDA), L-Glutamic acid N,N-diacetic acid, phosphoric acid, fumaric acid, tartaric acid, oxalic acid, maleic acid, malic acid, succinic acid and citric acid, and salts thereof. Preferred chelating agent is EDTA. In an exemplary embodiment, the EDTA can be ethylenediaminetetraacetic acid disodium salt.

The amount of the chelating agent in the composition may be 0.01% (w/v) to 5% (w/v), 0.05% (w/v) to 3% (w/v), or 0.07% (w/v) to 2.5% (w/v), 0.1% (w/v) to 2% (w/v), 0.1% (w/v) to 1% (w/v), or 0.1% (w/v) to 0.5% (w/v) by weight of chelating agent based on the volume of the composition. In preferred embodiments, the amount of chelating agent, for example EDTA, may be 0.05% to 0.5% (w/v), preferably 0.1% to 0.5% (w/v). In specific embodiments, EDTA may be absent.

### pH of the Compositions

The compositions will typically have a pH in the range 3.5 to 9, preferably 4.0 to 6.5, more preferably 5.0 to 6.0, which is a range at which many chemical drugs display maximum chemical stability. In compositions comprising dexamethasone, the pH is in the range of 4.0 to 6.0, preferably between 4.3 and 5.7, and more preferably between 4.5 and 5.5.

In some embodiments buffer salts may be included to control the pH of the aqueous compositions. Such buffer salts may include the following acids and their salts, by way of example and without limitation, acetic acid, sodium acetate, adipic acid, ammonium chloride, benzoic acid, sodium benzoate, boric acid, sodium bicarbonate, sodium borate, citric acid, glycine, maleic acid, monobasic sodium phosphate, dibasic sodium phosphate, HEPES, lactic acid, tartaric acid, potassium metaphosphate, potassium phosphate, monobasic sodium acetate, sodium bicarbonate, tris-hydroxymethylaminomethane, sodium tartrate, sodium citrate anhydrous and dehydrate, others known to those of ordinary skill in the art, salts of any of the above, and combinations thereof.

### Tonicity Agents

In certain embodiments, the compositions may comprise a tonicity adjusting agent that is used to make the composition essentially isotonic. The compositions will typically have osmolality of 200 to 450 milliosmoles per kilogram (mOsm/kg), more preferably 240 to 360 mOsm/kg. Examples of suitable tonicity adjusting agents include sodium chloride, potassium chloride, mannitol, sorbitol, dextrose, glycerin, and combinations thereof. Preferably, the electrolyte is sodium chloride.

The amount of tonicity adjusting agent in the composition of the disclosure may be 0.01 to 5% by weight of tonicity adjusting agent based on the volume of the composition. The concentration range may depend on the type of tonicity adjusting agent. For electrolytes like sodium chloride and potassium chloride the concentration range might be from 0.01% to 0.9% (w/v) or 0.1% to 0.7% (w/v) or 0.3% to 0.7%, while for non-electrolytes like mannitol and dextrose the range might be 0.1% to 5% (w/v).

### Antimicrobial Preservative

As described above, the aqueous compositions of the present disclosure comprise sorbic acid or/and its salts and include, by way of example and without limitation, potassium sorbate, sodium sorbate and calcium sorbate as antimicrobial preservative. The amount of the sorbic acid or its salts in the composition may be 0.01% (w/v) to 5% (w/v), in particular 0.1% (w/v) to 2% (w/v), more particularly 0.2% (w/v) to 1% (w/v), preferably 0.2% (w/v) to 0.8% (w/v) by weight of sorbic acid or sorbate salt on the volume of the composition.

The aqueous compositions of the present disclosure comprise only sorbic acid or sorbate salts as preservatives. No additional antimicrobial preservatives are added. Thus, the preservative that is used in the aqueous compositions of the present disclosure essentially consists of sorbic acid or sorbate salts. In some examples the antimicrobial preservative consists of sorbic acid or sorbate salts.

The antimicrobial preservative does not comprise a conventional topical ophthalmic antimicrobial agent selected from the group consisting of benzalkonium chloride, benzethonium chloride, butyl-para-hydroxybenzoate, chlorobutanol, methyl-para-hydroxybenzoate, polyguaternium-1, propyl-para-hydroxybenzoate, or oxidizing antimicrobial preservative agents including perborate, zinc or chlorite ions.

### Method of Production

The compositions of the instant disclosure may be prepared by suspending the individual components in water followed by heating in a closed container for about 20 min at 121° C. to form an essentially clear solution. Then the solution is allowed to cool to ambient temperature followed by equilibration at 22-23° C. under constant agitation. During the equilibration the pH of the compositions is adjusted to about 4.5 to about 7.5 with aqueous 0.1 N hydrochloric acid (HCl) solution and aqueous 1.0 N sodium hydroxide (NaOH) solution and the volume adjusted with distilled water. The preservative is added to the composition at any possible stage during production.

### Uses of the Composition

The ophthalmic compositions of the disclosure may be for use in the treatment of an ocular condition, in particular a posterior ocular condition, typically for drugs that are poorly-soluble in water, advantageously complexed with cyclodextrin as described above. In specific embodiments, the ophthalmic compositions of the disclosure may be for use in the treatment of pathological states that arise or are exacerbated by ocular angiogenesis and vascular leakage, for example, in diabetic retinopathy (including background diabetic retinopathy, proliferative diabetic retinopathy and diabetic macular edema); age-related macular degeneration (AMD) (including neovascular (wet/exudative) AMD, dry AMD, and Geographic Atrophy); pathologic choroidal neo vascularization (CNV) from any mechanism (i.e. high myopia, trauma, sickle cell disease; ocular histoplasmosis, angioid streaks, traumatic choroidal rupture, drusen of the optic nerve, and some retinal dystrophies); pathologic retinal neovascularization from any mechanism (i.e., sickle cell retinopathy, retinopathy of prematurity, Eales disease, ocular ischemic syndrome, carotid cavernous fistula, familial exudative vitreoretinopa thy, hyperviscosity syndrome, idiopathic occlusive arteriolitis, birdshot retinochoroidopathy, retinal vasculitis, sarcoidosis, or toxoplasmosis); uveitis; retinal vein occlusion (central or branch); ocular trauma; surgery induced edema; surgery induced neovascularization; cystoid macular edema; ocular ischemia; retinopathy of prematurity; Coats disease; sickle cell retinopathy and/or neovascular glaucoma.

The ophthalmic composition, and more specifically the composition comprising complex of dexamethasone/cyclodextrins including the preferred embodiments of the next section, may be used for the treatment of macular edema, such as diabetic macular edema. In this case, the ophthalmic composition may be topically administered to the eye in an amount of 1 drop of composition three times per day. The amount of active pharmaceutical ingredient, for example dexamethasone, in said composition may be from of 0.5 to 5% (w/v), preferably 1 to 4% (w/v), more preferably 1.0 to 3.0% (w/v) weight of active pharmaceutical ingredient based on the volume of the composition.

In other embodiments, the ophthalmic compositions of the disclosure may be for use in the treatment of an anterior ocular condition. The ophthalmic composition, and more specifically the composition comprising of dexamethasone/cyclodextrin complexes, including the preferred embodiments of the next section, may be used for inflammation following ocular surgery, typically following cataract surgery.

The present disclosure further concerns a method of treatment of eye disorders comprising topically administering to the eye, for example with a multi-dose eye dropper, a therapeutically efficient amount of the aqueous ophthalmic compositions as described herein.

The present disclosure also provides use of the aqueous ophthalmic compositions as described herein in the manufacture of a medicament for the treatment of eye disorders, in particular in the manufacture of a multidose pharmaceutical dosage form for the treatment of eye disorders.

The compositions of the disclosure do not need to be administered as frequently as known topical compositions. Indeed, due to the higher concentration of the active pharmaceutical ingredient in the composition and longer duration of delivery, the bioavailability of the active pharmaceutical ingredient in the posterior segment is significantly increased, so that a lower frequency of administration is possible, increasing patient compliance.

The present disclosure also covers the use of the compositions as an eye drop microsuspension, and in particular, multidose eye drop microsuspension, so that depending on the indication and its severity, respectively, the solutions may be administered instead of or in addition to ophthalmic injection solutions, thereby significantly enhancing patient compliance and clinical outcome.

### Preferred Embodiments

In a particularly preferred embodiment, the aqueous composition comprises:
1 to 4% of dexamethasone, for example 1.5% to 3% of dexamethasone;
1 to 35% of γ-cyclodextrin, for example 5 to 25% of γ-cyclodextrin;
2.2 to 2.8% of polymer, or 2.8 to 3.2%, for example 2.5% or 3.0% of polymer, typically poloxamer;
0 to 0.2% of stabilizing agent, for example 0.1% of stabilizing agent, typically, disodium edetate;
0 to 0.5% of an additive to prevent the oxidation of the corticosteroid, for example between 0.2% and 0.4%, or between 0.2% and 0.3%, of an additive to prevent the oxidation of the corticosteroid, typically phenolic antioxidants or reducing agents, such as water-soluble natural antioxidants, for example sodium thiosulfate, L-methionine, or 3,4-dihydroxybenzoic acid;
0 to 1% of tonicity adjusting agent, for example 0.57% of electrolyte, typically sodium chloride; and
water;
   wherein the % are % by weight based on the volume of the composition.

In a particular embodiment, an aqueous composition comprises or essentially consists of:
1 to 4% of dexamethasone, for example 1.5% to 3% of dexamethasone;
1 to 35% of γ-cyclodextrin, for example 5 to 25% of γ-cyclodextrin;
0 to 0.2% of stabilizing agent, for example 0.1% of stabilizing agent, typically, disodium edetate;
0 to 1% of tonicity adjusting agent, for example 0.57% of electrolyte, typically sodium chloride; and
water;
   wherein the % are % by weight based on the volume of the composition.

In a particular embodiment, an aqueous composition for use as described in the present specification comprises or essentially consists of;
1 to 4% of dexamethasone, for example 1.5% to 3% of dexamethasone;
1 to 35% of γ-cyclodextrin, for example 5 to 25% of γ-cyclodextrin;
optionally 2.2 to 2.8% of polymer or 2.8% to 3.2% of polymer, for example 2.5% or 3.0% of polymer, typically poloxamer;
0 to 0.2% of stabilizing agent, for example 0.1% of stabilizing agent, typically, disodium edetate;
0% to 0.8% of an additive to prevent the oxidation of the dexamethasone, for example between 0.1% and 0.5%, or between 0.2% and 0.4%, of an additive to prevent the oxidation of the corticosteroid, typically phenolic antioxidants or reducing agents, such as water-soluble natural antioxidants, and for example sodium thiosulfate, L-methionine, or 3,4-dihydroxybenzoic acid;
0 to 1% of tonicity adjusting agent, for example 0.57% of electrolyte, typically sodium chloride; and
water;
   wherein the % are % by weight based on the volume of the composition.

More specifically, a particularly preferred embodiment is an eye drop formulation comprising or essentially consisting of:
1.5% of dexamethasone;
14% of γ-cyclodextrin;
2.5% of poloxamer;
0 to 0.2% of stabilizing agent, for example 0.1% of disodium edetate;
0 to 1% of tonicity adjusting agent, for example 0.57% of sodium chloride;
0% to 0.6% of an additive to prevent the oxidation of the dexamethasone, for example between 0.2% and 0.4%, of an additive to prevent the oxidation of the corticosteroid, typically phenolic antioxidants or reducing agents, such as water-soluble natural antioxidants, for example sodium thiosulfate, L-methionine, or 3,4-dihydroxybenzoic acid;
   and
water;
   wherein the % are % by weight based on the volume of the composition.

Typically, an eye drop formulation has the following components:
1.5% of dexamethasone;
14% of γ-cyclodextrin;
2.5% of poloxamer;
between 0.0% and 0.1% of disodium edetate;
0.57% of sodium chloride;—between 0.0% and 0.5% of sodium thiosulfate; and
water.

Another particular embodiment is an eye drop formulation comprising or essentially consisting of:
3% of dexamethasone;
1 to 35% of γ-cyclodextrin, for example 20 to 25% of γ-cyclodextrin;
optionally 2.8 to 3.2% of polymer, for example 3.0% of polymer, typically poloxamer;
0 to 0.2% of stabilizing agent, for example 0.1% of stabilizing agent, typically, disodium edetate;
0% to 0.6% of an additive to prevent the oxidation of the dexamethasone, for example between 0.1% and 0.5%, or between 0.2% and 0.4%, of an additive to prevent the oxidation of the corticosteroid, typically phenolic antioxidants or reducing agents, such as water-soluble natural antioxidants, and for example sodium thiosulfate, L-methionine, or 3,4-dihydroxybenzoic acid;
0 to 1% of tonicity adjusting agent, for example 0.57% of electrolyte, typically sodium chloride; and
water;
   wherein the % are % by weight based on the volume of the composition.

Another particular embodiment is an eye drop formulation comprising or essentially consisting of:
3% of dexamethasone;
between 20 and 25% of γ-cyclodextrin;
optionally between 2.8 and 3.2% of poloxamer; for example 3.0% of poloxamer;
0 to 0.2% of stabilizing agent, for example 0.1% of disodium edetate;
0 to 1% of tonicity adjusting agent, for example 0.57% of sodium chloride;
0% to 0.6% of an additive to prevent the oxidation of the dexamethasone, for example between 0.1% and 0.5%, or between 0.2% and 0.4%, of an additive to prevent the oxidation of the corticosteroid, typically phenolic antioxidants or reducing agents, such as water-soluble natural antioxidants, and for example sodium thiosulfate, L-methionine, or 3,4-dihydroxybenzoic acid; and
water;
   wherein the % are % by weight based on the volume of the composition.

Typically, an eye drop formulation have the following components:
3% of dexamethasone;
between 20 and 25% of γ-cyclodextrin; for example 23% of γ-cyclodextrin;
between 2.8 and 3.2% of poloxamer;
0.1% of disodium edetate;
0.57% of sodium chloride; and
0.0% to 0.5% of sodium thiosulfate; typically 0% of sodium thiosulfate, and
water.

In a preferred embodiment of the method, the eye drop formulation for use in the above method, comprises or essentially consists of:
1.5% of dexamethasone;
14% of γ-cyclodextrin;
2.5% of poloxamer;
0 to 0.2% of stabilizing agent, for example 0.1% of disodium edetate;
0 to 1% of tonicity adjusting agent, for example 0.57% of sodium chloride;
0% to 0.6% of an additive to prevent the oxidation of the dexamethasone, for example between 0.1% and 0.5%, or between 0.2% and 0.4%, of an additive to prevent the oxidation of the corticosteroid, typically sodium thiosulfate;
   and
water;
   wherein the % are % by weight based on the volume of the composition.

The above composition embodiments are further characterized in that the final pH of the composition is comprised between 4.0 and 6.0, preferably between 4.3 and 5.7 and more preferably between 4.5 and 5.5.

The above composition embodiments are further characterized in that the amount of sorbic acid or its salts in the compositions may be 0.01% (w/v) to 5% (w/v), in particular 0.1% (w/v) to 2% (w/v), preferably 0.2% (w/v) to 1% (w/v), more preferably 0.2% (w/v) to 0.8% (w/v) by weight of sorbic acid or sorbate salt on the volume of the composition.

Also, the compositions comprise only sorbic acid or sorbate salts as preservatives. No additional antimicrobial preservatives are added. Thus, the antimicrobial preservative that is used in the aqueous compositions of the present disclosure essentially consists of sorbic acid or sorbate salts. In some examples the preservative consists of sorbic acid or sorbate salts.

The antimicrobial preservative does not comprise a conventional topical ophthalmic antimicrobial agent selected from the group consisting of benzalkonium chloride, benzethonium chloride, butyl-para-hydroxybenzoate, chlorobutanol, methyl-para-hydroxybenzoate, polyguaternium-1, propyl-para-hydroxybenzoate, or oxidizing antimicrobial preservative agents including perborate, zinc or chlorite ions.

The aqueous compositions of the present disclosure are preferably contained in a multidose eye drop container. A conventional three pieces ophthalmic container is made of three plastic parts assembled for ensuring bottle tightness: a bottle or core container, a tip or ophthalmic dropper enabling to distribute the liquid as a drop and a screwed cap enabling to open and close the whole container system. The whole system is usually made of polyolefin thermoplastic materials of different density and preferably of LDPE for both the container and the dropper (low density polyethylene). Prior to the very first opening of the container system, the container enables the preservation of the product integrity including its sterility. After the very first opening of the container, the prevention of the microbial contamination of the liquid formulation is ensured by the presence of the antimicrobial preservative in the composition, for a duration of about four weeks.

In specific embodiments, the multidose eye drop container of the present disclosure has a total volume of 1, 2, 5 or 10 ml. The bottle can be either partially filled or filled up to its maximum total volume in order to be adjusted of up to one month of the therapeutical treatment.

The multidose eye drop container comprises the aqueous compositions of the present disclosure. It preferably comprises the aqueous compositions of the above described preferred embodiments.

### Embodiments

Item 1. An aqueous composition comprising
drug/cyclodextrin complexes which comprise a drug and an alpha-, beta- or gamma-cyclodextrin; and
an antimicrobial preservative which comprises or essentially consists of sorbic acid or a pharmaceutically acceptable sorbic acid salt.

Item 2. The aqueous composition according to item 1, wherein the drug is selected from the group consisting of ace-inhibitors, kinase inhibitors, agents that influence basement membrane, agents that influence the growth of endothelial cells, adrenergic agonists or blockers, cholinergic agonists or blockers, aldose reductase inhibitors, analgesics, anesthetics, antiallergics, anti-inflammatory agents, steroidal anti-inflammatory agents, antihypertensives, antibacterials, antivirals, antifungals, antiprotozoals, anti-glaucoma agents, intraocular pressure lowering agents, prostaglandin analogs, anti-infective agents, antitumor agents, antimetabolites, and antiangiogenic agents, chloride channel activators, small-molecule inhibitors of the NLRP3 inflammasome for the treatment of inflammatory diseases, MCC-950, ecothiopathe iodine, and atropine, preferably dexamethasone.

Item 3. The aqueous composition according to items 1 or 2, wherein the cyclodextrin is gamma-cyclodextrin, preferably natural gamma-cyclodextrin.

Item 4. The aqueous composition according to any of items 1 to 3, wherein the pharmaceutically acceptable sorbic acid salts are selected from the group consisting of potassium sorbate, sodium sorbate and calcium sorbate.

Item 5. The aqueous composition according to any of items 1 to 4, wherein sorbic acid or pharmaceutically acceptable sorbic acid salts are comprised at a concentration of 0.01% (w/v) to 5% (w/v), preferably 0.1% (w/v) to 2% (w/v), more preferably 0.2% (w/v) to 1% (w/v), even more preferably 0.2% (w/v) to 0.8% (w/v) by weight of sorbic acid or sorbate salt based on the volume of the composition.

Item 6. The aqueous composition according to any of items 1 to 5, wherein alpha-, beta- or gamma-cyclodextrin are comprised at 1 to 35% (w/v), preferably 5 to 30% (w/v), more preferably 10 to 25% (w/v) by weight of cyclodextrin based on the volume of the composition.

Item 7. The aqueous composition according to any of items 1 to 6, which is a microsuspension comprising particles of said drug/cyclodextrin complexes, wherein from about 5% (w/v) to about 40% (w/v), preferably 10 to 30% (w/v), of the drug is in solution, as dissolved free drug or as dissolved drug/cyclodextrin complex(es), and from about 60% (w/v) to about 95% (w/v), preferably 70 to 90% (w/v) of the drug is in solid drug/cyclodextrin complex particles.

Item 8. The aqueous composition according to any of items 1 to 7, which is a microsuspension comprising particles of said drug/cyclodextrin complexes, and the average size D₅₀ of the particles in the solid phase is from about 1 μm to about 100 μm, typically from 1 μm to 50 μm, more preferably between 1 and 25 mm.

Item 9. The aqueous composition according to any of items 1 to 8, further comprising 0.01% (w/v) to 5% (w/v), preferably 0.05% (w/v) to 3% (w/v), more preferably 0.07% (w/v) to 2.5% (w/v), even more preferably 0.1% (w/v) to 2% (w/v) of a chelating agent as a stabilizer.

Item 10. The aqueous composition according to item 9, wherein the chelating agent is a divalent or polyvalent carboxylic acid.

Item 11. The aqueous composition according to item 10, wherein the chelating agent is selected from the group of ethylenediamine-tetraacetic acid (EDTA), 2,2′,2″-nitrilotriacetic acid (NTA), malic acid, maleic acid, succinic acid, and citric acid, preferably EDTA.

Item 12. The aqueous composition according to any of items 1 to 11, wherein the pH is in the range of 3.5 to 9, preferably 4.0 to 6.5, more preferably 5.0 to 6.0.

Item 13. The aqueous composition according to any of items 1 to 11, wherein the drug is dexamethasone, and wherein the pH is in the range of 4 to 6, preferably 4.5 to 5.7, more preferably 4.5 to 5.5.

Item 14. The aqueous composition according to any of items 1 to 13, wherein the composition comprises from about 0.1 to 5% (w/v), preferably 0.3% (w/v) to 3% (w/v), more preferably 0.5% (w/v) to 2% (w/v) of water soluble polymer.

Item 15. The aqueous composition according to any of items 1 to 14, further comprising one or more surface active polymers selected from the group of poloxamer, tyloxapol, polyalkyleneglycol, hydroxyalkylcellulose, hydroxyalkyl alkylcellulose, and polyvinyl alcohol are present.

Item 16. The aqueous composition according to any of items 1 to 15, further comprising a tonicity adjusting agent.

Item 17. The aqueous composition according to item 16, wherein the tonicity adjusting agent comprises sodium chloride.

Item 18. The aqueous composition according to item 17, wherein the composition comprises 0.01% (w/v) to 0.09% (w/v), preferably 0.1% to 0.7% (w/v), more preferably 0.3% to 0.7%, of sodium chloride.

Item 19. The aqueous composition according to any of items 1 to 18, wherein the aqueous composition does not comprise any other antimicrobial preservative agent.

Item 20. The aqueous composition according to any of items 1 to 19, wherein the aqueous composition does not comprise a topical ophthalmic antimicrobial agent selected from the group consisting of benzalkonium chloride, benzethonium chloride, butyl-para-hydroxybenzoate, chlorobutanol, methyl-para-hydroxybenzoate, polyguaternium-1, propyl-para-hydroxybenzoate, or oxidizing antimicrobial preservative agents including perborate, zinc or chlorite ions.

Item 21. The aqueous composition according to any of items 1 to 20, which comprises or essentially consists of:
1 to 4% of dexamethasone; for example 1.5% or 3% of dexamethasone,
1 to 35% of γ-cyclodextrin, for example 5 to 25% of γ-cyclodextrin;
0.01% (w/v) to 5% (w/v), preferably 0.1% (w/v) to 2% (w/v), more preferably 0.2% (w/v) to 1% (w/v) by weight of sorbic acid or sorbate salt;
optionally 2.2 to 2.8% of polymer or 2.8% to 3.2% of polymer, for example 2.5% or 3.0% of polymer, typically poloxamer, and preferably poloxamer 407;
0 to 0.2% of stabilizing agent, for example 0.1% of stabilizing agent, typically, disodium edetate;
0 to 0.8% of an additive to prevent the oxidation of the dexamethasone, for example between 0.1% and 0.5%, or between 0.2% and 0.4%, of an additive to prevent the oxidation of the corticosteroid, typically phenolic antioxidants or reducing agents, such as water-soluble natural antioxidants, and more preferably sodium thiosulfate;
0 to 1% of tonicity adjusting agent, for example 0.57% of electrolyte, typically sodium chloride;
a pH comprised between 4.5 and 5.5, and
water;
   wherein the % are % by weight based on the volume of the composition.

Item 22. The aqueous composition according to item 21, which is a suspension comprising solid complexes of dexamethasone and gamma-cyclodextrin.

Item 23. The aqueous composition according to any of items 21 or 22, which is a microsuspension, preferably comprising 80% to 95% of the corticosteroid in microparticles having a diameter of 1 μm to 10 μm.

Item 24. The aqueous composition according to any of items 1 to 23, which is a multidose ophthalmic composition.

Item 25. A multidose eye drop container, comprising an aqueous composition of any one of items 1 to 24, preferably a volume comprised between 2 mL and 10 mL, and more preferably between 2.5 mL and 5 mL.

Item 26. The aqueous composition according to any of items 1 to 24, for use in the topical treatment of retinal diseases.

Item 27. The aqueous composition according to any of items 1 to 25, for use in treating a condition of posterior segment and/or the anterior segment of the eye.

Item 28. The aqueous composition for use according to item 26, wherein said condition is selected from the group of age-related macular degeneration (AMD), diabetic retinopathy (DR), diabetic macular edema (DME), retinopathy of prematurity, pathologic choroidal neo vascularization (CNV) and inflammation following ocular surgery, typically following cataract surgery.

Item 29. A method for treating a condition of the posterior segment and/or the anterior segment of the eye in a subject in need thereof, said method comprising applying topically to the eye surface of said subject, an aqueous composition according to any one of items 1 to 24, comprising a drug as the active principle, in an amount which delivers a therapeutically effective amount of said drug to said segment or segments of the eye.

Item 30. A method for avoiding microbial contamination of an eye drop formulation comprising cyclodextrin, said method comprising adding an antimicrobial preservative efficient amount of sorbic acid or its pharmaceutically acceptable salts in said eye drop formulation.

Item 31. The method of item 30, wherein said eye drop formulation is contained in a multidose eye dropper, which is preferably made of polyolefin thermoplastic materials of different density, preferably of LDPE.

Item 32. A process for manufacturing an eye drop formulation comprising cyclodextrin, said process comprising
(i) providing an eye drop formulation comprising drug/cyclodextrin complexes which comprise a drug and an alpha-, beta- or gamma-cyclodextrin; and
(ii) adding an antimicrobial preservative efficient amount of sorbic acid or its pharmaceutically acceptable salts in said eye drop formulation.

### Examples

In view of the above description and the examples below, one of ordinary skill in the art will be able to practice the invention as claimed without undue experimentation. The examples given should not be considered exhaustive, but merely illustrative of only a few of the many embodiments contemplated by the present invention.

### Measuring Methods

### Particle Size Diameter D₅₀

The D₅₀ diameter of a particle, such as a solid complex of active pharmaceutical ingredient and cyclodextrin is also known as the median diameter or the medium value of the particle size distribution. Diameter D50 corresponds to the value of the particle diameter at 50% in the cumulative distribution. For example, if D₅₀ is 5 μm, then 50% of the particles in the sample are larger than 5 μm, and 50% smaller than 5 μm. Diameter D₅₀ is usually used to represent the particle size of a group of particles.

The diameter D50 is measured by laser diffraction particle size analysis. For particle size of complexes comprising an active pharmaceutical ingredient, the particle size is measured by laser diffraction particle size analysis according to Pharm. Eur. 2.9.31 (Particle size analysis by laser diffraction, January 2010) applying the following parameters:

Mastersizer method description:

| Instrument | Malvern Mastersizer 3000 Hydro MV | |
|---|---|---|
| Software | Mastersizer v 3.70 | |
| Particle type | Opaque particle (Fraunhofer approximation) | |
| Dispersant | Type I water | |
| Refractive index of the dispersant | 1.33 | |
| Background measurements duration | 10 | sec |
| Sample measurements duration | 1 | sec |
| Number of measurements | 15 | |
| Obscuration lower limit | 2% | |
| Obscuration higher limit | 20% | |
| Measurement mode | Auto-start with stabilization time of 0.2 sec | |
| Stirrer speed | 1200 | rpm |
| Analysis model | General purpose | |
| Sensitivity | Normal, with “fine powder” mode on | |
| Result type | Volume distribution | |
| Sample preparation | Manual homogenization by shaking | |
| Sample size | 500 | μl |

| | |
|---|---|
| Cleaning between measurements | Triple rinsing with tap water and once with type |
| | II water |

Analysis of samples with Olympus BX43 light microscope was done in accordance with Pharm. Eur. 2.9.37. 1 μl of manually homogenized eye drops was scanned under different magnification (up to 40×). Treatment of microscopic photos was done by means of equipped Olympus LC30 digital camera and LCmicro v2.2 software.

### Percentage of Drug in Solid Complex and Percentage of Dissolved Drug

The amount of drug in the form of solid complexes and the amount of dissolved drug is obtained by centrifuging the composition at 6000 rpm at a temperature of 22-23 degrees Celsius for 20-30 minutes.

The amount of dissolved drug corresponds to the amount of drug in the supernatant as measured by high-performance liquid chromatography.

The percentage of drug in the form of a solid complex is obtained with the following formula:

$$\% drug in solid complex = \frac{( total drug - dissolved drug )}{total drug} \times 100$$

wherein
“total drug” is the total amount of drug introduced in the composition in mg/mL; and
“dissolved drug” is the amount of drug in the supernatant in mg/mL.

The percentage of dissolved drug is obtained with the following formula:

$$\% dissolved drug = 100 - \% drug in solid complex$$

### Example 1: Antimicrobial Preservative System Screening

Several antimicrobial preservative agents commonly used and approved for topical ophthalmic drugs from a regulatory standpoint are tested head-to-head with an ophthalmic suspension of dexamethasone including the natural γ-cyclodextrin. As the pH of the formulation may influence the efficacy of the antimicrobial activity, all the samples are set-up at pH 5.0, which is the target pH of the formulation ensuring its best stability.

The concentration of each evaluated preservative agent is set-up at its maximum accepted limit from a regulatory standpoint, according to the US-FDA database (“Inactive Ingredient list”).

### Antimicrobial Preservative Agents Information:

| | | | | | Known |
|---|---|---|---|---|---|
| | | | | | suitable |
| | | | | | pH |
| | | | | | for |
| | | | Water | Temper- | anti- |
| | | | solubility | ature | microbial |
| ID | CAS-RN | MW | (at 20° C.) | Stability | activity |
| BAK | 63449-41-2 | 283.88 | High (75%) | Stable | 4-10 |
| BZT | 121-54-0 | 448.10 | Good (5-18%) | Stable | 4-10 |
| CBT | 57-15-8 | 177.46 | Low (0.8%) | <65° C. | 4-10 |
| PQD | 75345-27-6 | 557/2500 | Good (>40%) | Stable | 4-10 |
| SA | 110-44-1 | 112.13 | Low (0.15%) | Low | 4-5 |
| KS | 24634-61-5 | 150.2 | Good (>50%) | Medium | 4-5 |
| PB | | | | | |
| BPB | 94-26-8 | 194.23 | Low (0.25%) | Yes | 4-8 |
| MPB | 99-76-3 | 152.15 | | | 4-8 |
| PPB | 94-13-3 | 180.2 | | | 4-8 |

BAK: Benzalkonium chloride
BZT: Benzethoniuym chloride
PQD: Polyquad ® (Polyquaternium-1)
CBT: Clorobutanol
SA: Sorbic acid
KS: Potassium sorbate
Parabens (PB): BPB: butyl paraben, MPB: methyl paraben, PPB: propyl paraben

### Formulation Table:

| | Sample 1 | Sample 2 | Sample 3 | Sample 4 | Sample 5 | Sample 6 | Sample 7 | Sample 8 |
|---|---|---|---|---|---|---|---|---|
| | Base formulation | | | | | | | |
| | Same base composition for all samples: | | | | | | | |
| | (Dexamethasone⁽¹⁾ (base): 1.5%/Gamma-Cyclodextrin⁽¹⁾: 14%/Poloxamer | | | | | | | |
| Antimicrobial | 407⁽¹⁾: 2.5%/Sodium thiosulfate pentahydrate^{(1):} 0.471%/EDTA⁽¹⁾: 0.10%/ | | | | | | | |
| preservatives | Sodium chloride⁽¹⁾: 0.57%/Purified water⁽¹⁾: Q.S. 100 g/Final pH 5.0) | | | | | | | |
| BAK⁽³⁾ | n/a | 0.03% | n/a | n/a | n/a | n/a | n/a | n/a |
| BZT⁽¹⁾ | n/a | n/a | 0.02% | n/a | n/a | n/a | n/a | n/a |
| PQD | n/a | n/a | n/a | 0.001% | n/a | n/a | n/a | n/a |
| CBT⁽¹⁾ | n/a | n/a | n/a | n/a | 1.1% | n/a | n/a | n/a |
| Paraben mix⁽²⁾ | n/a | n/a | n/a | n/a | n/a | See⁽²⁾ | n/a | n/a |
| SA⁽¹⁾ | n/a | n/a | n/a | n/a | n/a | n/a | 0.2% | n/a |
| KS⁽¹⁾ | n/a | n/a | n/a | n/a | n/a | n/a | n/a | 0.47% |

⁽¹⁾USP-NF material quality grade
% ⇔ %_{w/w} ⇔ equivalent to g/100 g
⁽²⁾Paraben mix: EP/USP-NF/Butyl paraben: 0.05% + Methyl paraben: 0.02% + Propyl paraben: 0.2%
⁽³⁾BAK grade: EP/USP-NF/Alkyl moieties content: 65.8% C₁₂, 34.2% C₁₄, 0.0% C₁₆

### Antimicrobial Preservative Efficacy Methodology:

For assessing the antimicrobial preservative agents, an Antimicrobial Efficacy Test (AET) is performed according to both USP-NF and EP standards
USP-NF: “American Pharmacopoeia” monograph <51>
EP: “European Pharmacopoeia” monograph <50103>

Those AETs consist in challenging the drug formulation containing an antimicrobial preservative agent at a certain concentration with specific microbial strains. The results are provided in microbial reduction expressed in logarithmic units. The acceptance criteria are specific of each standard (either US or EU) according to the table below. For being declared as passing the test, all the criteria must be achieved over the 28 days period. If at least one criterion is missed, then the whole test is declared failed.

AET acceptance criteria (microbial burden reduction in Log units)

| | Reading time point: | | | | |
|---|---|---|---|---|---|
| | 6 hours | 24 hours | 7 days | 14 days | 28 days |
| EU | * NLT 2 Log | * NLT 3 Log | * n/a | n/a | * No Recovery |
| Pseudomonas | (Crit A.) | (Crit A.) | (Crit A.) | | (Crit A.) |
| aeruginosa | * n/a | * NLT 1 Log | * NLT 3 Log | | * No Increase |
| | (Crit. B) | (Crit. B) | (Crit. B) | | (Crit. B) |
| USA | n/a | n/a | NLT 1 Log | NLT 3 Log | No Increase |
| EU | * NLT 2 Log | * NLT 3 Log | * n/a | n/a | * No Recovery |
| Staphylococcus | (Crit A.) | (Crit A.) | (Crit A.) | | (Crit A.) |
| aureus | * n/a | * NLT 1 Log | * NLT 3 Log | | * No Increase |
| | (Crit. B) | (Crit. B) | (Crit. B) | | (Crit. B) |
| USA | n/a | n/a | NLT 1 Log | NLT 3 Log | No Increase |
| EU | n/a | n/a | * NLT 2 Log | * n/a | * No Increase |
| Candida | | | (Crit A.) | (Crit A.) | (Crit A.) |
| albicans | | | * n/a | * NLT 1 Log | * No Increase |
| | | | (Crit. B) | (Crit. B) | (Crit. B) |
| USA | n/a | n/a | No Increase | No Increase | No Increase |
| EU | n/a | n/a | * NLT 2 Log | * n/a | * No Increase |
| Aspergillus | | | (Crit A.) | (Crit A.) | (Crit A.) |
| brasiliensis | | | * n/a | * NLT 1 Log | * No Increase |
| | | | (Crit. B) | (Crit. B) | (Crit. B) |
| USA | n/a | n/a | No Increase | No Increase | No Increase |
| EU | n/a | n/a | n/a | n/a | n/a |
| Escherichia | | | | | |
| coli | | | | | |
| USA | n/a | n/a | NLT 1 Log | NLT 3 Log | No Increase |

NLT: Not Less Than
n/a: Not Applicable
In EU, if criteria A is not matched, then criteria B can be acceptable with some additional justifications

Based on current knowledge those three microbial challenge tests (AETs), also known in the past as preservative efficacy tests (PET), are known to be more or less difficult to pass. EP/A test is known to be the most difficult one to pass. EB/B is less difficult than EB/A. USP-NF test is usually less difficult or sometimes equivalent to EP/B.

Antimicrobial Preservative Efficacy Results (pH 5.0):

| | Sample | Sample | Sample | Sample | Sample | Sample | Sample | Sample |
|---|---|---|---|---|---|---|---|---|
| | 1 | 2 | 3 | 4 | 5 | 6 | 7 | 8 |
| Preservative | PF⁽¹⁾ | BAK | BZT | PQD | CBT | PBN | SA | KS |
| AET results | Fail | Fail | Fail | Fail | Fail | Fail | PASS | PASS |
| (USP) | | | | | | | | |
| AET results | Fail | Fail | Fail | Fail | Fail | Fail | Fail | Fail |
| (EP) | | | | | | | | |

⁽¹⁾Preservative Free

It can be concluded from the example that none of the tested antimicrobial agents are effective with the dexamethasone formulation including γ-cyclodextrin with the exception of the sorbate derivatives: sorbic acid and potassium sorbate.

The sorbate derivatives are able to match the acceptance criteria of the USP-NF AET but not those of the EP standard with respect to the formulation of this example.

### Example 2: Sorbate Derivatives (pH 4 to 6)

Two formulations of a dexamethasone ophthalmic suspension are prepared at two different pH values: pH 4.0 and pH 6.0. For each formulation, two different sorbate derivative agents are evaluated for efficacy in order to compare their possible antimicrobial behavior according to the USP-NF methodology.

### Formulation Table and AET Results:

| | Sample | Sample | Sample | Sample |
|---|---|---|---|---|
| | 9 | 10 | 11 | 12 |
| Base | Same base composition for all samples: | | | |
| formulation | (Dexamethasone⁽¹⁾ (base): 1.5%/Gamma-Cyclodextrin⁽¹⁾: | | | |
| | 14%/Poloxamer 407⁽¹⁾: 2.5%/Sodium thiosulfate | | | |
| | pentahydrate^{(1):} 0.471%/EDTA⁽¹⁾: 0.10%/Sodium | | | |
| | chloride⁽¹⁾: 0.57%/Purified water⁽¹⁾: Q.S. 100 g/ | | | |
| | Hydrochloric acid or sodium hydroxide Q.S. targeted pH) | | | |
| SA⁽¹⁾ | 0.2% | 0.2% | n/a | n/a |
| KS⁽¹⁾ | n/a | n/a | 0.47% | 0.47% |
| Final pH | 4.0 | 6.0 | 4.0 | 6.0 |
| AET results | Pass | Fail | Pass | Fail |
| (USP-NF | | (close | | (close |
| methodology) | | to pass) | | to pass) |

⁽¹⁾USP-NF material quality grade

It can be concluded that both sorbic acid and potassium sorbate have a similar antimicrobial preservative profile, with the same activity in the tested pH range. For both compounds, the antimicrobial activity is influenced by the pH. This antimicrobial activity matches the USP-NF at a pH below 6.0, usually between 4.0 and 5.7, and more preferably between 4.5 and 5.5.

### Example 3: Stability Stress Test

Example 2 indicates that sorbate derivatives can be utilized as an antimicrobial preservative system in a γ-cyclodextrin containing ophthalmic composition. In order to assess the stability behavior of the sorbate derivatives in such a composition a temperature stress test is performed on two compositions including either sorbic acid or potassium sorbate. The test consists in autoclaving the composition in a glass container, applying three consecutive cycles of 121° C./20 min. Such a temperature exposure is not representative of customary storage conditions for such a product, but it can be used for voluntary degrading the formulation and in order to possibly detect some differences between the tested samples. The test also includes one “preservative free” sample as a reference point (Reference Sample 1).

### Formulation Table:

| | | | Reference |
|---|---|---|---|
| | Sample 13 | Sample 14 | Sample 1 |
| Base | Same base composition for all samples: | | |
| formulation | (Dexamethasone⁽¹⁾ (base): 1.5%/Gamma-Cyclodextrin⁽¹⁾: 14%/ | | |
| | Poloxamer 407⁽¹⁾: 2.5%/Sodium thiosulfate pentahydrate^{(1):} 0.471%/ | | |
| | EDTA⁽¹⁾: 0.10%/Sodium chloride⁽¹⁾: 0.57%/Purified water⁽¹⁾: Q.S. 100 g/ | | |
| | Final pH 5.0) | | |
| SA⁽²⁾ | 0.2% | n/a | n/a |
| KS⁽³⁾ | n/a | 0.47% | n/a |

⁽¹⁾USP-NF material quality grade
⁽²⁾Sorbic acid
⁽³⁾Potassium sorbate

Stability Results (after 3× Heat Exposure (Three Autoclaving Cycles);

| | ID | Sample 13 | Sample 14 | Sample 1 |
|---|---|---|---|---|
| Product Quality | Test Acceptance | SA (0.2%) | KS (0.47%) | Preservative |
| Attributes | Criteria | | | Free |
| Product aspect | White | Complies | Complies | Complies |
| | homogeneous | | | |
| | suspension | | | |
| pH | NMT 10% pH | Complies | Complies | Complies |
| | unit change | (+5.5%) | (+6.9%) | (+3.0%) |
| Osmolality | NMT 10% change | Complies | Complies | Complies |
| | (mOsmol/kg) | (+3.7%) | (+3.0%) | (+2.4%) |
| Dexamethasone | NMT 5% change | Complies | Complies | Complies |
| assay (HPLC) | | (−1.4%) | (−1.9%) | (−3.1%) |
| Preservative assay | NMT 30% change | FAILS | Complies | n/a |
| (HPLC) | | (−41.6%) | (−29.9%) | |
| STS assay | NMT 30% change | Complies | Complies | Complies |
| (HPLC) | | (−11.2%) | (−16.4%) | (−6.3%) |
| Dexamethasone | NMT 10% content | Complies | Complies | Complies |
| Impurity alpha | | (6.6%_{w/v}) | (6.6%_{w/v}) | (5.4%_{w/v}) |
| (HPLC) | | | | |
| Total | NMT 1% content | Complies | Complies | Complies |
| unidentified | | (0.4%_{w/v}) | (0.6%_{w/v}) | (0.7%_{w/v}) |
| dexamethasone | | | | |
| impurities | | | | |
| (HPLC) | | | | |

NMT: Not More Than
STS: Sodium Thiosulfate
Impurity alpha: Main degradation product of dexamethasone after thermal exposure

It appears that sorbic acid and potassium sorbate have a slightly different stability profile in the γ-cyclodextrin formulation at pH 5 in a glass container. Potassium sorbate appears to be more stable in this specific example that includes dexamethasone.

The stability profile of potassium sorbate is satisfying and matches all the acceptance criteria. It is noted that the two sorbate derivatives have a small stabilizing effect on dexamethasone used as the drug-substance in this specific example (with a little more of the main impurity but with less drop of the total dexamethasone content).

From a stability standpoint, both sorbic acid and potassium sorbate can be used in a γ-cyclodextrin containing composition. For such composition additionally including dexamethasone potassium sorbate is preferred.

### Example 4: Minimum Effective Dose Determination for Potassium Sorbate

| | Sample 15 | Sample 16 | Sample 17 | Sample 18 | Sample 14 |
|---|---|---|---|---|---|
| Antimicrobial | 0.047%_{w/v} | 0.117%_{w/v} | 0.235%_{w/v} | 0.352%_{w/v} | Maximum |
| preservative | ( [symbol] Y − 90%) | ( [symbol] Y − 75%) | ( [symbol] Y − 50%) | ( [symbol] Y − 25%) | “Regulatory” |
| (Potassium | | | | | dose |
| Sorbate) | | | | | 0.47% (=Y) |
| concentration | | | | | |
| Formulation | | | | | |
| Base formulation | Dexamethasone (base): 1.5%/Gamma-Cyclodextrin: 14%/Poloxamer 407: | | | | |
| | 2.5%/Sodium thiosulfate pentahydrate: 0.47%/EDTA⁽¹⁾: 0.10%/Sodium | | | | |
| | chloride: 0.57%/Purified water: Q.S. 100 g/Final pH 5.0 | | | | |
| AET-USP/NF <51> | Fail | Fail | Pass | Pass | Pass |

As a conclusion for the above described formulation of dexamethasone eye drops, the minimum effective dose of potassium sorbate used as an antimicrobial agent is 0.235% w/v (according to the USP-NF AET methodology).

### Example 5: Influence of Some Formulation Excipients

The here above described eye drop formulation includes two excipients intended to specifically stabilize the dexamethasone active ingredient: the sodium thiosulfate (STS) used as antioxidant agent, and the EDTA used as stabilizing agent.

EDTA is pharmaceutical excipient also known to improve the efficacy of some antimicrobial preservative agents. EDTA is not enough powerful for being considered as an antimicrobial preservative agent by itself, but it is sometimes considered as a preservative aid, in example when used with either quaternary ammonium agents or parabens. On the contrary, with some other antimicrobial preservative agents like mercurial derivatives, including thimerosal and phenylmercuric nitrate, the antimicrobial preservative activity is reduced in presence of EDTA (Handbook of Pharmaceutical excipients, 6^{th} Ed., Pharmaceutical Press editions, 2009, “Edetic acid” monograph).

STS and EDTA have been included in the example of the dexamethasone formulation for specifically improving the long term storage stability of this corticoid over time. Nevertheless, in the case of formulation with other active compound than a corticoid, it would be helpful to know whether those two excipients may have an influence on the antimicrobial preservative activity of the formulation.

To investigate the matter, three samples including potassium sorbate used at its maximum allowed concentration have been designed for evaluating the impact of STS and EDTA on the antimicrobial preservative efficacy provided by the sorbate derivative.

| | Sample 19 | Sample 20 | Sample 21 |
|---|---|---|---|
| Base formulation | Dexamethasone(base): 1.5%/Gamma-Cyclodextrin: | | |
| | 14%/Poloxamer 407: 2.5%/Potassium Sorbate: 0.47%/Sodium | | |
| | chloride: 0.57%/Purified water: Q.S. 100 g/Final pH 5.0 | | |
| EDTA | 0.1% | n/a | n/a |
| STS | n/a | 0.471% | n/a |
| AET-USP/NF <51> | Pass | Pass | Pass |
| AET - EP/A | Fail | Fail | Fail |
| AET - EP/B | Pass | Pass | Pass |

The results demonstrate that neither EDTA nor STS have an improving influence on the antimicrobial preservative activity of potassium sorbate. Surprisingly, in absence of at least one of those compounds, the antimicrobial preservative activity of the sorbate derivative is even improved: criteria B requirements of the EP/AET are met, which was not the case in the presence of those two excipients (USP/AET criteria met in this case only).

As a result, formulation of liquid formulation including a drug/gamma-cyclodextrin complex can be preserved from a microbiology standpoint by sorbic acid or one of its salts. Additionally it can be noticed that the antimicrobial activity of the sorbate derivative is inversely proportional to the dose of either STS or EDTA. The lower the EDTA concentration is in the formulation containing a drug/cyclodextrin complex, the higher the antimicrobial preservative efficacy of the sorbate derivative will be. Thus, the present disclosure provides an aqueous composition having a reduced dosed of either STS or EDTA while also showing improved antimicrobial preservative activity.

The above examples show that sorbic acid and potassium sorbate exhibit a superior antimicrobial preservation effect at a low preservative concentration to aqueous eye drop microsuspension containing solid dexamethasone/cyclodextrin complexes in presence of various pharmaceutical excipients. The following are examples showing that the antimicrobial preservation effect is maintained when dexamethasone is replaced by other active pharmaceutical ingredients (APIs) such as dorzolamide, everolimus and latanoprost.

### Example 6: Aqueous Dorzolamide/Cyclodextrin Microsuspension

The composition of the aqueous dorzolamide/cyclodextrin microsuspension formulation was as follows: 1.1% (w/v) dorzolamide hydrochloride (Curia, Spain), 7.0% (w/v) gamma-cyclodextrin, 0.1% (w/v) EDTA, 0.05% (w/v) tyloxapol, 0.5% (w/v) hydroxypropyl methylcellulose (Metolose 90SH-4000SR), 0.47% (w/v) potassium sorbate, hydrochloric acid Q.S. to pH 5.0, and water (USP Type 1) Q.S. The pH of the final product was 4.99 and 64% of the drug was in solid drug/cyclodextrin microparticles. The formulation passed the Antimicrobial Efficacy Test (AET) of the USP/NF and failed AET-EP/A and AET-EP/B.

### Example 7: Aqueous Everolimus/Cyclodextrin Microsuspension

The composition of the aqueous everolimus/cyclodextrin microsuspension was as follows: 0.025% (w/v) everolimus (BrightGene, China), 10% (w/v) gamma-cyclodextrin, 0.1% (w/v) EDTA, 0.1% (w/v) tyloxapol, 0.47% (w/v) potassium sorbate, hydrochloric acid Q.S. to pH 5.0, and water (USP Type 1) Q.S. The pH of the final product was 5.01 and 62% of the drug was in solid drug/cyclodextrin microparticles. The formulation passed both the AET of the USP/NF and the B-test of EP and failed the AET A-test of EP.

### Example 8: Aqueous Latanoprost/Cyclodextrin Microsuspension

The composition of the aqueous latanoprost/cyclodextrin microsuspension was as follows: 0.005% (w/v) latanoprost (Chemodex Ltd., Switzerland), 1.6% (w/v) gamma-cyclodextrin, 0.1% (w/v) EDTA, 0.1% (w/v), 0.25% (w/v) hydroxypropyl methylcellulose (Metolose 90SH-4000SR), 0.5% (w/v) sodium chloride, 0.47% (w/v) potassium sorbate, hydrochloric acid Q.S. to pH 5.0, and water (USP Type 1) Q.S. The pH of the final product was 5.00 and 8% of the drug was in solid drug/cyclodextrin microparticles. The formulation passed both the AET of the USP/NF and the B-test of EP and failed the AET A-test of EP.

We claim:

1. An aqueous ophthalmic composition comprising:
1% to 4% of dexamethasone;
1% to 35% of γ-cyclodextrin;
0.01% to 5% of sorbic acid or a sorbate salt;
0 to 0.2% of a stabilizing agent;
0 to 0.8% of an antioxidant;
0 to 1% of an electrolyte;
water;
wherein the % are % by weight based on the volume of the aqueous ophthalmic composition;
wherein the aqueous ophthalmic composition comprises a microsuspension comprising solid complexes of dexamethasone and γ-cyclodextrin; and
wherein the aqueous ophthalmic composition has a pH of between 4.5 and 5.5.

2. The aqueous ophthalmic composition of claim 1, wherein the composition comprises 1.5% to 3% of dexamethasone.

3. The aqueous ophthalmic composition of claim 1, wherein the composition comprises 5% to 25% of γ-cyclodextrin.

4. The aqueous ophthalmic composition of claim 1, wherein the composition comprises 0.1% to 2% of sorbic acid or the sorbate salt.

5. The aqueous ophthalmic composition of claim 1, wherein the composition comprises 0.2% to 1% of sorbic acid or the sorbate salt.

6. The aqueous ophthalmic composition of claim 1, wherein the composition further comprises 2.2% to 2.8% of a poloxamer.

7. The aqueous ophthalmic composition of claim 1, wherein the composition further comprises about 2.5% of a poloxamer.

8. The aqueous ophthalmic composition of claim 1, wherein the composition further comprises 2.8% to 3.2% of a poloxamer.

9. The aqueous ophthalmic composition of claim 1, wherein the composition further comprises about 3.0% of a poloxamer.

10. The aqueous ophthalmic composition of claim 1, wherein the composition comprises about 0.1% of the stabilizing agent.

11. The aqueous ophthalmic composition of claim 1, wherein the stabilizing agent is disodium edetate.

12. The aqueous ophthalmic composition of claim 1, wherein the composition comprises 0.1% to 0.5% of the antioxidant.

13. The aqueous ophthalmic composition of claim 1, wherein the composition comprises 0.2% to 0.4% of the antioxidant.

14. The aqueous ophthalmic composition of claim 1, wherein the antioxidant is sodium thiosulfate.

15. The aqueous ophthalmic composition of claim 1, wherein the electrolyte is sodium chloride.

16. The aqueous ophthalmic composition of claim 1, wherein the composition comprises:
1.5% to 3% of dexamethasone;
5% to 25% of γ-cyclodextrin;
0.1% to 2% of sorbic acid or a sorbate salt;
about 0.1% of disodium edetate;
0.1% to 0.5% of sodium thiosulfate;
0 to 1% of sodium chloride;
water;
wherein the % are % by weight based on the volume of the aqueous ophthalmic composition;
wherein the aqueous ophthalmic composition comprises a microsuspension comprising solid complexes of dexamethasone and γ-cyclodextrin; and
wherein the aqueous ophthalmic composition has a pH of between 4.5 and 5.5.

17. The aqueous ophthalmic composition of claim 16, wherein the composition comprises 0.2% to 1% of sorbic acid or the sorbate salt.

18. The aqueous ophthalmic composition of claim 16, wherein the composition further comprises 2.2% to 2.8% of a poloxamer.

19. The aqueous ophthalmic composition of claim 16, wherein the composition further comprises about 2.5% of a poloxamer.

20. The aqueous ophthalmic composition of claim 16, wherein the composition further comprises 2.8% to 3.2% of a poloxamer.

21. The aqueous ophthalmic composition of claim 16, wherein the composition further comprises about 3.0% of a poloxamer.

22. The aqueous ophthalmic composition of claim 16, wherein the composition comprises 0.2% to 0.4% of sodium thiosulfate.

23. A method of treating a retinal disease comprising administering an effective amount of the aqueous ophthalmic composition of claim 1 to a human patient in need thereof.

24. The method of claim 23, wherein the retinal disease is age-related macular degeneration (AMD).

25. The method of claim 23, wherein the retinal disease is diabetic retinopathy (DR).

26. The method of claim 23, wherein the retinal disease is diabetic macular edema (DME).

27. The method of claim 23, wherein the retinal disease is retinopathy of prematurity (ROP).

28. The method of claim 23, wherein the retinal disease is pathologic choroidal neo vascularization (CNV).

29. The method of claim 23, wherein the retinal disease is inflammation following an ocular surgery.

30. The method of claim 29, wherein the ocular surgery is cataract surgery.
